(19) United States
(12) Patent Application Publication
(10) Pub. No.: US 2003/0027174 A1
(43) Pub. Date: Feb. 6, 2003

(54) Identification of nucleotide sequences specific for mycobacteria and development of differential diagnosis strategies for mycobacterial species

(76) Inventors: Jean-Luc Gala, St.Stevens-Woluwe (BE); Pascal Vannuffel, Buvrinnes (BE)

Correspondence Address: NIXON & VANDERHYE P.C., 8th Floor, 1100 North Glebe Road, Arlington, VA, 22201, US

(21) Appl. No.: 10/074,246

(22) Filed: Feb. 14, 2002

Related U.S. Application Data
(60) Provisional application No. 60/269,848, filed on Feb. 21, 2001.
(60) Provisional application No. 60/292,509, filed on May 23, 2001.

(51) Int. Cl. C12Q 1/68

(52) U.S. Cl. 435/6

(57) ABSTRACT

The present invention relates to methods and devices for detecting and differentiating between Mycobacterium strains in a sample based upon species-specific upstream p34 gene region (us-p34) sequences. New us-p34 sequences and probes and primers derived therefrom are provided as well as methods and diagnostic kits based on the same.

The present application claims benefit of U.S. Provisional applications 60/269,848, filed Feb. 21, 2001, and 60/292,509, filed May 23, 2001, the entire contents of each of which are incorporated herein by reference.

### FIELDS OF THE INVENTION

The present invention refers to new genetic sequences, diagnostic and/or quantification methods and devices using said sequences for the identification of various types of Mycobacterium strains

### BACKGROUND TO THE INVENTION.

Classification

The mycobacterial diseases may be divided in three categories: tuberculosis which is caused either by M. tuberculosis or M. bovis, both belonging to the M. tuberculosis-complex (TUB), leprosy caused by M. leprae, and diseases caused by nontuberculous mycobacteria (NTM), which cause all mycobacterial diseases other than tuberculosis and leprosy. Historically, tuberculosis and leprosy are the two preponderant human mycobacterial diseases. In recent years however, NTM have become more frequent in developed countries, partly because of the development of Acquired Immunodeficiency Syndrome (AIDS) (1). Tuberculosis occurs only in humans and animals, no other reservoir has been found to date. Conversely, the reservoir of mycobacteria responsible for NTM disease is mainly environmental and the majority of these mycobacteria are naturally resistant to known antimycobacterial drugs, making eradication unfeasible. Identification of mycobacteria species by rapid and specific methods is now mandatory. In particular, there is a need for rapid differentiation of strict human and animal pathogenic mycobacteria (such as, for example, M. tuberculosis-complex, M. paratuberculosis, M. leprae) from potentially pathogenic mycobacteria (such as, for example, M. avium-complex; M. chelonae, M. kansasii, M. xenopi, M. simiae, M. malmoense) and from normally saprophytic species (such as, for example, M. gordonae; M. terrae; M. nonchromogenicum; M. flavescens; M. gastri; M. smegmatis). Except for the strict pathogens, the majority of mycobacteria can indeed be found everywhere in the nature (soil, fresh and seawater) and can colonise temporary or permanently the human respiratory or digestive tract after ingestion or inhalation.

a) Members of the tuberculosis-complex (TUB):

This group comprises M. tuberculosis, M. bovis, M. africanum, M. microti.

b) Non tuberculous mycobacteria (NTM):

Members of the M. avium-complex (MAC-complex) includes several environmental species that resemble M. avium and M. intracellulare or that have intermediate characteristics common to these species (M. avium; M. intracellulare; M. paratuberculosis, M. scrofulaceum).

Mycobacteria other than TUB (also named MOTT) and not belonging to the MAC-complex (such as, for example, M. malmoense, M. sulzgai, M. kansasii, M. xenopi, M. chelonae, M. simiae, M. marinum, M. gordonae, M. forfuitum).

Epidemiology

Tuberculosis infects one-third of the world's population and kills more than 3 million of people each year. Tuberculosis, caused by mycobacteria of the M. tuberculosis group (TUB), is still highly endemic in large parts of the world and also the potential for introduction into and further transmission within countries where it has become rare (2). Tuberculosis has indeed re-emerged in many industrialised nations in recent years as a results of AIDS and changing population dynamics characterised by increased numbers of migrant workers, immigrants, and homeless people (3). Of particular concern is the spread of drug-resistant strains (4). Cases of tuberculosis are more and more often difficult to treat because of the increasing prevalence of TUB strains that are resistant to all front-line antituberculous drugs. In HIV-1 infected people, TUB is the most common opportunistic bacterial infections. In advanced stages of AIDS, mycobacterial infections due to members of the M. avium-intracellulare complex (MAC) are the most common systemic bacterial opportunistic infection (1). Moreover, reduced and compromised immune function as found in new-borns, infants, and immune-suppressed individuals allows opportunistic infections caused by mycobacteria other than M. tuberculosis (NTM) including M. avium-intracellulare, M. chelonae, M. fortuitum, M. kansasii, M. xenopi, M. marinum, M. ulcerans, M. scrofulaceum, and M. szulgai.

The increasing number of mycobacterial infections has made it clinically important to quickly identify mycobacteria at the species level. The diagnosis of a pathogenic versus a non-pathogenic species not only has epidemiological implications but is also relevant to the demands of patient management. Individuals with highly contagious infections may be isolated to prevent the spread of the disease. As a matter of fact, antibiotic treatments may vary according to the species encountered. Today, the number of non-tuberculous infections is difficult to assess because there is no system for notification as it exists for M. tuberculosis. The currently reported frequency of these species is likely to be an underestimation due to the lack of additional testing in cases of minimal disease or misidentification as M. tuberculosis. Despite the likely underestimation of NTM diseases, a growing number of NTM isolates are submitted to laboratories for identification. This may reflect an increase in the prevalence of opportunistic mycobacterial disease (notably in the AIDS context), or it may also reflect an increase in the number and nature of specimens submitted for culture brought about by a greater awareness of tuberculosis.

The ability to detect and identify these mycobacterial diseases in an early stage would potentially decrease morbidity and mortality and lessen its socio-economic impact. The <<gold standard>> for definite diagnosis has remained culturing the mycobacterium combined with phenotypic tests (for example, the analysis of fafty acid and mycolic acid contents). However, traditional culture methods for human specimens can require up to 8 weeks (this varies according to the mycobacterial species). For instances, primary culture of M. ulcerans under optimal conditions may take several months (5).

Other methods based on molecular biology technologies have allowed the development of genetic tests for the identification of some mycobacterial species. In the last decade, the use of restriction fragment length polymorphism (RFLP) analysis, oligotyping, and nucleic acid probes have been proposed as a mean for detecting and definitively identifying infectious agents (6). These tests are highly specific and rapid when compared to conventional culture method and have been applied to a wide variety of pathogens, especially to fastidious micro-organisms like mycobacteria. To date, the design of molecular tests has speeded up the diagnosis of this fastidious genus but still suffers some drawbacks. For species identification, a highly polymorphic region of the 16S rRNA gene has been shown to contain species-specific polymorphisms. This region is currently used in several commercially available assays, applicable either directly on a specimen (Accu-Probe; Gen-Probe) or after enzymatic amplification of the target for improved sensitivity (AMTD; Gen-Probe; Amplicor MTB, Roche). However, these commercial kits are mostly designed for the diagnosis of the most common disease-causing mycobacterial species, i.e. M. tuberculosis and MAC strains, and each kit has been designed to identify only one mycobacterial species per test which makes it a very expensive and limitative way of identifying mycobacterial species.

### SUMMARY OF THE INVENTION

The newly identified and characterised sequences described in the present invention enable the design of differential diagnosis strategies. These diagnosis strategies are able to identify, in one single assay, a wide range of mycobacterial species that include TUB and NTM. In addition, within the NTM group, it is even possible to differentiate between the members of the MAC-complex. Moreover, these molecular targets open new perspectives for the quantification of mycobacteria strains in clinical samples. The present invention aims to provide new genetic sequences, methods and devices for the improvement of the identification and/or the quantification of various types of mycobacteria species through their upstream-p34 (us-p34) determinants, which allow by a rapid molecular screening, their epidemiological study as well as their rapid characterisation in clinical human, animal and/or environmental samples. Another aim of the invention is to identify similar genetic sequences which may exist in known and yet unknown Mycobacteria species.

### DETAILED DESCRIPTION OF THE INVENTION

The present inventors for the first time recognised, by sequencing the upstream part of the p34 gene, species-specific differences in the sequences between various mycobacterial strains, allowing determination of the presence and identification of specific Mycobacterium strains or species in various samples. Therefore, according to a first embodiment the present invention relates to a method for detecting at least one Mycobacterium strain in a sample, comprising:

(i) providing at least one Mycobacterium species-specific upstream p34 gene region (us-p34) nucleotide probe,

(ii) reacting said us-p34 nucleotide probe with said sample under conditions that allow for the selective formation of nucleotide duplexes between said us-p34 nucleotide probe and a corresponding Mycobacterium nucleic acid target present in said sample, and,

(iii) detecting any nucleotide duplexes containing said us-p34 nucleotide probe.

Said probes may comprise the whole or only parts of species-specific us-p34 gene region sequences, some of which are disclosed in FIG. 3 which the skilled man has aligned in FIGS. 1 and 8 to 10. On the basis of such an alignments it is within the skilled man's knowledge to actually design suitable probes to be used in said method. In this method more than one Mycobacterium strain may be detected, provided that the species-specific probes can be distinguished from each other. Numerous well-known methods are described in the art to come to this result. As an example, the species-specific probes can be labelled with different markers which can be detected by various methods or devices after hybridisation with the target as to distinguish between the nucleotide duplexes formed.

It should be undestood that the above described methode is not limited to the us-p34 sequences actually provided in FIG. 3. The us-p34 sequences envisaged in the invention encompass the complete us-p34 sequences and also all the corresponding sequences of the various Mycobacerium species which are shown to contain species-specific differences.

Said differences not only comprise point mutations but also include deletion and/or insertion of several nucleotide bases. These deletions and/or insertions may result in different lengths of the us-p34 upstream region between various species. According to a preferred embodiment the present invention more particularly relates to a method as defined above, wherein said Mycobacterium species-specific us-p34 nucleotide probe specifically hybridizes with at least part of a sequence selected from FIG. 3, or the complement thereof, or the corresponding sequences wherein T has been replaced by U.

The specific or non-specific hybridization of the species-specific us-p34 probes is further illustrated in FIG. 5.

In Tables 3 (a) to (c), nucleotide sequences are represented for use as probe or primer in various methods for detection, identification, hybridisation to, capture, amplification of Mycobacterium species-specific nucleic acids.

The present invention more particularly relates to a method as defined above, wherein said Mycobacterium species-specific us-p34 nucleotide probe is selected from the group of sequences represented by SEQ ID NOs 8 to 54 (Tables 3 (a) to (c)) and SEQ ID NOs 57 to 74 (FIG. 3), or the corresponding sequences wherein T has been replaced by U. According to yet another embodiment, the invention relates to a method for the differential detection of Mycobacteria in a sample, comprising:

(i) providing at least two distinct Mycobacterium species-specific us-p34 nucleotide probes,

(ii) reacting said us-p34 nucleotide probes with said sample under conditions that allow for the selective formation of nucleotide duplexes between said us-p34 specific nucleotide probe and a Mycobacterium nucleic acid present in said sample,

(iii) detecting any nucleotide duplexes containing said us-p34 nucleotide probe, and,

(iv) inferring from the nucleotide duplex formed, the presence and the identification of a specific Mycobacterium strain.

In a preferred embodiment, said species-specif us-p34 probes are chosen from the group of sequences represented by SEQ ID NOs 8 to 54 and 57 to 74. The present invention also relates to a method for detecting at least one Mycobacteria strain in a sample, comprising:

(i) providing at least one suitable us-p34 primer pair comprising a sense and anti-sense us-p34 primer,

(ii) reacting said us-p34 primer pair with said sample under conditions that allow for the selective amplification of us-p34 sequences of at least one Mycobacterium nucleic acid present in said sample,

(iii) detecting the amplified product of step (ii), and,

(iv) inferring from the amplified product formed, the presence and the identification of at least one (specific) Mycobacterium strain or species.

It should be clear to the one skilled in the art and from the information provided in the Figures, especially FIG. 4, that the above described method does not specifically require the use of a Mycobacterium species-specific us-p34 primers to be able to identify specific Mycobacterium strans or species from the amplification product formed. It has been found by the present inventors that the differences in the us-p34 region of various species results in different lengths of the amplification products when amplified for instance with the universal primers provided in Tables 1 and 2 (SEQ ID NOs 1 to 7). The detection of Mycobacterium species in a sample is therefore possible through means of non-specific or specific amplification of the us-p34 regions. Said universal primers are especially designed by the present inventors to amplify us-p34 sequences of most if not all Mycobacterium species. In Tables 1 and 2 the skilled man will find several universal sense and antisense primers which can be used according to the sheme represented in FIG. 2 to form a suitable primer pair for amplifying the desired us-p34 region in the above method.

Alternatively also species-specific primers can be used in such a method, which may lead to the species-specific identification of the Mycobacterium present in the sample. Species-specific us-p34 primers are derived from us-p34 sequences (parts of the sequences as given in FIG. 3) which are present in one specific species but not in the others. On the basis of the alignment of the sequences as given in FIG. 3 (see FIGS. 8 to 10), it is within the knowledge of the skilled man to design actual primer sequences which will selectively amplify one species (type) of Mycobacterium. Nucleotides having a sequence as respresented in any of SEQ ID NOs 8 to 54 (Table 3 (a) to (c)) are used as a species-specific primer in a such a method.

The invention thus also relates to a method for detecting at least one Mycobacterium strain in a sample, comprising:

(i) providing at least one suitable primer pair comprising a sense or antisense Mycobacterium species-specific us-p34 primer selected from the group of sequences represented in SEQ ID NOs 8 to 54,

(ii) reacting said us-p34 primer pair with said sample under conditions that allow for the selective amplification of an us-p34 sequence in a Mycobacterium nucleic acid present in said sample, and,

(iii) detecting the amplified product of step (ii), and,

(iv) inferring from the amplification product the presence and the identification of at least one specific Mycobacterium strain.

It should be obvious for the skilled in the art that the second primer constituting said primer pair can be chosen from the sequences provided in Table 3 (a) to (c) where sequences of a sense and antisense primer are provided for a specific Mycobacterium species. Alternatively, the skilled in the art can choose the second primer from the universal primers presented in Tables 1 and 2 and represented by SEQ ID NOs 1 to 7. As such, the amplification products resulting from one universal and one species-specific primer are still species-specific.

The invention also relates to a method for the differential detection of mycobacteria in a sample, comprising:

(i) providing at least one suitable us-p34 primer pair containing a sense and anti-sense us-p34 primer,

(ii) reacting said us-p34 primer pair with said sample under conditions that allow for the selective amplification of us-p34 sequences of at least one Mycobacterium nucleic acid present in said sample,

(iii) detecting the amplified product of step (ii), and,

(iv) inferring from the amplified product formed, the presence and the identification of at least one (specific) Mycobacterium

As described above, already the length of the amplification products may be indicative for the presence of a specific Mycobacterium species. Therefore according to a preferred embodiment, the primers to be used in the above method are selected from the sequences represented in SEQ ID NOs 1 to 7 and presented as universal primers in Tables 1 and 2. Alternatively, also species-specific us-p34 primers are prefebly used in the above method. Therefore the invention also related to a method as described above wherein the primers are selected from the species-specific us-p34 primers represented by SEQ ID NOs 8 to 54. The invention also relates to a method as described above wherein a first primer constituting the primer pair is selected from the universal primers represented by SEQ ID NOs 1 to 7 and a second primer is selected from the species-specific primers represented by SEQ ID NOs 8 to 54.

According to a preferred embodiment the invention relates to a method for the detection of MAC complex Mycobacterium species in a sample, comprising:

(i) providing at least one us-p34 probe selected from the group of sequences represented in SEQ ID NOs 8, 14, 15, 22, 27, 28, 29, 34, 35, 50, 51, 57, 68, and 73,

(ii) reacting said us-p34 probe with said sample under conditions that allow for the selective formation of nucleotide duplexes between said us-p34 nucleotide probe and a MAC complex Mycobacterium nucleic acid target in said sample, and,

(iii) detecting any nucleotide duplexes containing said us-p34 nucleotide probe.

Members belonging to the MAC comples Mycobacterium species are presented in Table 4. In an alternative method also embodied by the present invention, step (iii) of the above-described method can be replaced by the step of determining the us-p34 sequence of said MAC Mycobacterium nucleic acid in said sample wherein the with the expression “determining the us-p34 sequence” is meant, for instance, direct sequencing to confirm the presence of said specific MAC complex us-p34 sequence. Other methods can be by mass spectrometry, capillary electrophoresis or HPLC.

According to a preferred embodiment the invention relates to a method for the detection of MOTT Mycobacterium species in a sample, comprising:

(i) providing at least one us-p34 probe selected from the group of sequences represented in SEQ ID NOs 9 to 13, 16 to 21, 24, 25, 26, 30 to 33, 36 to 47, 49, 53, 54, 59 to 64, 67, 69 to 72 and 74,

(ii) reacting said us-p34 primer probe said sample under conditions that allow for the selective formation of nucleotide duplexes between said us-p34 nucleotide probe and a MOTT Mycobacterium nucleic acid target in said sample, and,

(iii) detecting any nucleotide duplexes containing said us-p34 nucleotide probe. Members belonging to the MOTT comples Mycobacterium species are presented in Table 4. In an alternative method also embodied by the present invention, step (iii) of the above-described method can be replaced by the step of determining the us-p34 sequence of said MOTT Mycobacterium nucleic acid in said sample wherein the with the expression “determining the us-p34 sequence” is meant, for instance, direct sequencing to confirm the presence of said specific MOTT complex us-p34 sequence. Other methods can be by mass spectrometry, capillary electrophoresis or HPLC. The invention further relates to a method for detecting new us-p34 sequences in a sample, comprising:

(i) providing at least one suitable primer pair comprising a sense and anti-sense us-p34 primer selected from the sequences represented in SEQ ID NOs 1 to 7,

(ii) reacting said us-p34 primer pair with said sample under conditions that allow for the amplification of an us-p34 sequence in a Mycobacterium nucleic acid target in said sample, and,

(iii) determining the sequence of the amplification product obtained in (ii).

The expression “new us-p34 sequences” refers to new (not yet identified) mycobacterium species sequences. Some of these new sequences are disclosed in FIG. 3.

The present invention further relates to a method for the differential detection of mycobacteria in a sample, comprising:

(i) providing at least one suitable primer pair comprising a sense and anti-sense us-p34 primer selected from SEQ ID NOs 1 to 7,

(ii) reacting said us-p34 primer pair with said sample under conditions that allow for the amplification of an us-p34 sequence in a Mycobacterium nucleic acid target in said sample,

(iii) selectively hybridizing the amplification products obtained in (ii) with at least one Mycobacterium species-specific us-p34 nucleotide probe selected from the group of sequences represented in SEQ ID NOs 8 to 74,

(iv) detecting any nucleotide duplexes containing said Mycobacterium species-specific us-p34 nucleotide probe, and,

(v) inferring from the nucleotide duplex formed the presence of a specific Mycobacterium species.

Different examples of how universal primers and species-specific and probes are used in the above method are shown in Table 3.

The present invention also relates to a Mycobacterium species-specific us-p34 nucleotide probe or primer comprising at least 8 contiguous nucleotides from one of the nucleic acid sequences represented in SEQ ID NOs 57 to 74, or the complement thereof, or the corresponding sequences wherein T has been replaced by U.

The present invention also relates to the Mycobacterium species-specific us-p34 nucleotide probe or primer as represented in SEQ ID NOs 8 to 54. The invention also relates to the Mycobacterium us-p34 universal primers as represeted in SEQ ID NOs 1 to 7.

The present invention also relates to the non-tuberculosis Mycobacteria species-specific us-p34 nucleotide probe as defined above selected from Table 3. The present invention also relates to a nucleic acid comprising a sequence selected from Table 3 and FIG. 3 and having a sequence as represented by SEQ ID NOs 8 to 54. Preferably said sequence is a continuous sequence of any of said SEQ ID Nos having a length from 8 to maximally the full length of said SEQ ID NOs.

The present invention also relates to a composition comprising at least one nucleotide probe, primer or sequence as defined above. Said composition preferably contains two, three or more of said components.

The present invention also relates to a diagnostic kit comprising a probe, primer or sequence as defined above or a composition as defined above.

The present invention also relates to a solid support for the detection of (specific) mycobacteria comprising fixed to said support at least two capture probes selected from Table 3 (SEQ ID NOs 8 to 54) or from the sequences represented by SEQ ID NOs 57 to 74.

The invention also relates to a solid support for the detection of mycobacteria comprising fixed to said support at least one nucleic acid having a sequence as represented in SEQ ID NOs1to7.

The present invention also relates to a solid support as defined above for use in a method as defined above.

Different techniques can be applied to perform the methods of the present invention. These techniques may comprise immobilizing the target polynucleic acids, after amplification, on a solid support and performing hybridization with labelled oligonucleotide probes. Alternatively, the probes of the invention may be immobilized on a solid support (covalently or non-covalently) and hybrdization may be performed with labelled target polynucleic acids, possibly after amplification. This technique is called reverse hybridization. Said techniques are well-known to the man skilled in the art. It is to be understood that also any other technique for detection of the above-mentioned amplified target sequences are also covered by the present invention. Such a technique can involve sequencing or other micro-array methods known in the art. In preferred methods according to the invention it is possible to differentiate between two Mycobacterium species.

Therefore in a preferred embodiment, the invention relates to a method for differentiating between Mycobacterium bovis and Mycobacterium tubercolusosis in a sample, comprising:

(i) providing at least one us-p34 probe selective for Mycobacterium bovis or Mycobacterium tuberculosis wherein said probe is SEQ ID NO 66 for Mycobacterium bovis or SEQ ID NO 65 for Mycobacterium tuberculosis,

(ii) reacting said us-p34 nucleotide probe with said sample under conditions that allow for the selective formation of nucleotide duplexes between said us-p34 nucleotide probe and a corresponding Mycobacterium bovis or tuberculosis nucleic acid target present in said sample, and,

(iii) detecting any nucleotide duplexes containing said Mycobacterium bovis or tuberculosis specifc us-p34 nucleotide probe.

The invention also relates to a method for differentiating between Mycobacterium bovis and Mycobacterium tubercolusosis in a sample, comprising:

(i) providing at least one suitable primer pair comprising at least one sense or antisense us-p34 primer selective for Mycobacterium bovis or Mycobacterium tuberculosis wherein said primer is SEQ ID NO 66 for Mycobacterium bovis or SEQ ID NO 65 for Mycobacterium tuberculosis,

(ii) reacting said us-p34 primer pair with said sample under conditions that allow for the selective amplification of Mycobacterium bovis and/or Mycobacterium tuberculosis nucleic acid target present in said sample, and,

(iii) inferring from the reaction product(s) the presence of Mycobacterium bovis and/or Mycobacterium tuberculosis in said sample.

The invention further relates to a method for differentiating between Mycobacterium avium and Mycobacterium avium subspecies paratuberculosis in a sample, comprising:

(i) providing at least one us-p34 probe selective for Mycobacterium avium and Mycobacterium avium subspecies paratuberculosis wherein said probe is selected from the sequences represented in SEQ ID NOs 8, 27 to 29, 50 or 58 for Mycobacterium avium or SEQ ID NO 68 for Mycobacterium avium subspecies paratuberculosis;

(ii) reacting said us-p34 nucleotide probe with said sample under conditions that allow for the selective formation of nucleotide duplexes between said us-p34 nucleotide probe and a corresponding Mycobacterium avium and Mycobacterium avium subspecies paratuberculosis nucleic acid target present in said sample, and,

(iii) detecting any nucleotide duplexes containing said Mycobacterium avium and Mycobacterium avium subspecies paratuberculosis specifc us-p34 nucleotide probe

The invention also relates to a method for differentiating between Mycobacterium avium and Mycobacterium avium subspecies paratuberculosis in a sample, comprising:

(i) providing at least one suitable primer pair comprising at least one sense or antisense us-p34 primer selective for Mycobacterium avium and Mycobacterium avium subspecies paratuberculosis wherein said primer is selected from the sequences represented in SEQ ID NOs 8, 27 to 29, 50 or 58 for Mycobacterium avium or SEQ ID NO 68 for Mycobacterium avium subspecies paratuberculosis,

(ii) reacting said us-p34 primer pair with said sample under conditions that allow for the selective amplification of Mycobacterium avium and Mycobacterium avium subspecies paratuberculosis nucleic acid target present in said sample, and,

(iii) inferring from the reaction product(s) the presence of Mycobacterium avium and Mycobacterium avium subspecies paratuberculosis in said sample.

The following definitions and explanations will permit a better understanding of the present invention.

The target material in the samples to be analysed may either be DNA or RNA, e.g. genomic DNA, messenger RNA or amplified versions thereof. These molecules are in this application also termed “polynucleic acids” or “nucleic acids”. Well-known extraction and purification procedures are available for the isolation of RNA or DNA from a sample (e.g. in Sambrook et al., 1989).

The term “probe” according to the present invention refers to a single-stranded oligonucleotide which is designed to specifically hybridize to the target polynucleic acids. According to the invention those probes can be as long as the complete us-p34 sequences the Mycobacterium p34 gene. Some examples of those sequences are provided in the figures and in the nucleic acids having a sequence as represented in SEQ ID NOs 57 to 74. Also preferred according to the invention are probes having a sequence which is part of the us-p34 upstream sequences of about 5 to 50 nucleotides long, more preferably from about 10 to 25 nucleotides. Particularly preferred lengths of probes include 10, 11, 12, 13, 14, 15, 16, 17, 18, 19, 20, 21, 22, 23, 24 or 25 nucleotides. The nucleotides as used in the present invention may be ribonucleotides, deoxyribonucleotides and modified nucleotides such as inosine or nucleotides containing modified groups which do not essentially alter their hybridization characteristics. Probes may be labelled with a label of choice (e.g. biotin, fluoresceine, radioactive labels) according to methods well known in the art.

The term “primer” according to the present invention refers to a single stranded oligonucleotide sequence capable of acting as a point of initiation for synthesis of a primer extension product which is complementary to the nucleic acid strand to be copied. The length and the sequence of the primer must be such that they allow to prime the synthesis of the extension products. Preferably the primer is about 5-50 nucleotides long. Specific length and sequence will depend on the complexity of the required DNA or RNA targets, as well as on the conditions at which the primer is used, such as temperature and ionic strength. It is to be understood that the primers of the present invention may be used as probes and vice versa, provided that the experimental conditions are adapted.

The expression “suitable primer pair” in this invention refers to a pair of primers allowing specific amplification of a us-p34 target polynucleic acid fragment as defined above. Said amplification can be species-pecific or not, dependending on the use of universal or species-specific primers as indicated in the description.

The term “target region” of a probe or a primer according to the present invention is a sequence within the polynucleic acids to be detected to which the probe or the primer is completely complementary or partially complementary (i.e. with some degree of mismatch). It is to be understood that the complement of said target sequence is also a suitable target sequence in some cases.

“Specific hybridization” of a probe to a target region of a polynucleic acid means that said probe forms a duplex with part of this region or with the entire region under the experimental conditions used, and that under those conditions said probe does not form a duplex with other regions of the polynucleic acids present in the sample to be analysed.

“Specific hybridization” of a primer to a target region of a polynucleic acid means that, during the amplification step, said primer forms a duplex with part of this region or with the entire region under the experimental conditions used, and that under those conditions said primer does not form a duplex with other regions of the polynucleic acids present in the sample to be analysed. It is to be understood that “duplex” as used hereby, means a duplex that will lead to specific amplification.

The fact that amplification primers do not have to match exactly with the corresponding target sequence in the template to warrant proper amplification is amply documented in the literature. However, when the primers are not completely complementary to their target sequence, it should be taken into account that the amplified fragments will have the sequence of the primers and not of the target sequence. Primers may be labelled with a label of choice (e.g. biotin). The amplification method used can be either polymerase chain reaction (PCR), ligase chain reaction (LCR), nucleic acid sequence-based amplification (NASBA), transcription-based amplification system (TAS), strand displacement amplification (SDA) or amplification by means of Qβ replicase or any other suitable method to amplify nucleic acid molecules known in the art.

Probes and primer sequences are represented throughout the specification as single stranded DNA oligonucleotides from the 5′ to the 3′ end. It is obvious to the man skilled in the art that any of the below-specified probes can be used as such, or in their complementary form, or in their RNA form (wherein T is replaced by U).

The probes, primer and other nucleotide sequences according to the invention can be prepared by cloning of recombinant plasmids containing inserts including the corresponding nucleotide sequences, if need be by excision of the latter from the cloned plasmids by use of the adequate nucleases and recovering them, e.g. by fractionation according to molecular weight. The probes and primers according to the present invention can also be synthesized chemically, for instance by the conventional phospho-triester method.

The oligonucleotides used as primers or probes may also comprise nucleotide analogues such as phosphorothiates, alkylphosphorothiates or peptide nucleic acids or may contain intercalating agents. As most other variations or modifications introduced into the original DNA sequences of the invention these variations will necessitate adaptations with respect to the conditions under which the oligonucleotide should be used to obtain the required specificity and sensitivity. However the eventual results of hybridization will be essentially the same as those obtained with the unmodified oligonucleotides. The introduction of these modifications may be advantageous in order to positively influence characteristics such as hybridization kinetics, reversibility of the hybrid-formation, biological stability of the oligonucleotide molecules, etc.

The term “solid support” can refer to any substrate to which an oligonucleotide probe can be coupled, provided that it retains its hybridization characteristics and provided that the background level of hybridization remains low. Usually the solid substrate will be a microtiter plate, a membrane (e.g. nylon or nitrocellulose) or a microsphere (bead) or a chip (biochip). Prior to application to the membrane or fixation it may be convenient to modify the nucleic acid probe in order to facilitate fixation or improve the hybridization efficiency. Such modifications may encompass homopolymer tailing, coupling with different reactive groups such as aliphatic groups, NH₂ groups, SH groups, carboxylic groups, or coupling with biotin, haptens or proteins.

The term “labelled” refers to the use of labelled nucleic acids. Labelling may be carried out by the use of labelled nucleotides incorporated during the polymerase step of the amplification or labelled primers, or by any other method known to the person skilled in the art. The nature of the label may be isotopic (³²p, 35S, etc.) or non-isotopic (biotin, digoxigenin, etc.).

The term “sample” refers any sample of biological or non-biological origin. The term “biological sample or sample” refers to for instance naso-pharyngeal aspirates, throat or nasopharyngeal swabs, nasopharyngeal washes or tracheal aspirates or other respiratory tract sample comprising DNA or RNA. Samples of non-biological origin can be from any organic or inorganic material which can be suspected to contain microbial material.

For designing probes with desired characteristics, the following useful guidelines known to the person skilled in the art can be applied.

Because the extent and specificity of hybridization reactions such as those described herein are affected by a number of factors, manipulation of one or more of those factors will determine the exact sensitivity and specificity of a particular probe, whether perfectly complementary to its target or not. The importance and effect of various assay conditions are explained further herein.

The stability of the [probe:target] nucleic acid hybrid should be chosen to be compatible with the assay conditions. This may be accomplished by avoiding long AT-rich sequences, by terminating the hybrids with G:C base pairs, and by designing the probe with an appropriate Tm. The beginning and end points of the probe should be chosen so that the length and %GC result in a Tm about 2-10° C. higher than the temperature at which the final assay will be performed. The base composition of the probe is significant because G-C base pairs exhibit greater thermal stability as compared to A-T base pairs due to additional hydrogen bonding. Thus, hybridization involving complementary nucleic acids of higher G-C content will be more stable at higher temperatures.

Conditions such as ionic strength and incubation temperature under which a probe will be used should also be taken into account when designing a probe. It is known that the degree of hybridization will increase as the ionic strength of the reaction mixture increases, and that the thermal stability of the hybrids will increase with increasing ionic strength. On the other hand, chemical reagents, such as formamide, urea, DMSO and alcohols, which disrupt hydrogen bonds, will increase the stringency of hybridization. Destabilization of the hydrogen bonds by such reagents can greatly reduce the Tm. In general, optimal hybridization for synthetic oligonucleotide probes of about 10-50 bases in length occurs approximately 5° C. below the melting temperature for a given duplex. Incubation at temperatures below the optimum may allow mismatched base sequences to hybridize and can therefore result in reduced specificity.

It is desirable to have probes which hybridize only under conditions of high stringency. Under high stringency conditions only highly complementary nucleic acid hybrids will form; hybrids without a sufficient degree of complementarity will not form. Accordingly, the stringency of the assay conditions determines the amount of complementarity needed between two nucleic acid strands forming a hybrid. The degree of stringency is chosen such as to maximize the difference in stability between the hybrid formed with the target and the non-target nucleic acid.

Regions in the target DNA or RNA which are known to form strong internal structures inhibitory to hybridization are less preferred. Likewise, probes with extensive self-complementarity should be avoided. As explained above, hybridization is the association of two single strands of complementary nucleic acids to form a hydrogen bonded double strand. It is implicit that if one of the two strands is wholly or partially involved in a hybrid that it will be less able to participate in formation of a new hybrid. There can be intramolecular and intermolecular hybrids formed within the molecules of one type of probe if there is sufficient self complementarity. Such structures can be avoided through careful probe design. By designing a probe so that a substantial portion of the sequence of interest is single stranded, the rate and extent of hybridization may be greatly increased. Computer programs are available to search for this type of interaction. However, in certain instances, it may not be possible to avoid this type of interaction.

Standard hybridization and wash conditions are disclosed in the Materials & Methods section of the Examples. Other conditions are for instance 3X SSC (Sodium Saline Citrate), 20% deionized FA (Formamide) at 50° C. Other solutions (SSPE (Sodium saline phosphate EDTA), TMAC (Tetramethyl ammonium Chloride), etc.) and temperatures can also be used provided that the specificity and sensitivity of the probes is maintained. When needed, slight modifications of the probes in length or in sequence have to be carried out to maintain the specificity and sensitivity required under the given circumstances.

The term “hybridization buffer” means a buffer allowing a hybridization reaction between the probes and the polynucleic acids present in the sample, or the amplified products, under the appropriate stringency conditions.

The term “wash solution” means a solution enabling washing of the hybrids formed under the appropriate stringency conditions.

The invention, now being generally described, will be more readily understood by reference to the following examples and figures, which are included merely for the purposes of illustration of certain aspects and embodiments of the present invention and are in no way to be construed as limiting the present invention. All of the references mentioned herein are incorporated by reference.

### FIGURE LEGENDS

FIG. 1. Multiple nucleotide sequences alignment of M. bovis (MB), M. tuberculosis (MT), M. avium subsp. paratuberculosis (MPT) and M. avium ss (MA) us-p34 genes. Gaps between sequences are indicated by dots (.). Vertical bar (I) indicates identity across sequences. The start codon (ATG) for the p34 ORF is in bold. Primer sequences are indicated by shaded boxes. The arrows indicate point mutations between M. tuberculosis and M. bovis and M. avium subsp. paratuberculosis and M. avium ss.

FIG. 2. Amplifications of us-p34 regions with primers U1, U2, U3, U4, U6 and U9.

FIG. 3. New us-p34 sequences (5′ to 3′). Primers used to obtain the sequence (either U2-U1 U3-U1; U4-U1; U2-U9; U3-U9 or U4-U9) and the amplicon size are as indicated. Sequence variations (point mutations) found in the same species (for instance M. ulcerans) are also indicated when known.

FIG. 4. U1-U4 consensus amplification of us-p34 regions of different mycobacterial species.

FIG. 5. Specific and non-specific hybridization.

FIG. 6. Differential reverse hybridization of mycobacteria target amplicons on a nylon membrane disclosing species-specific mycobacteria probes.

a) Unlabeled amplified DNA segments specific for various mycobacteria species were first transferred on nylon membrane (M. tuberculosis (TB), M. avium (AV), M. szulgai (SZ), M. kansasii (KA), M. xenopi (XE), M. simiae (SI) and M. malmoense (ML)).

b) Digoxigenin-labeled amplicons from M. tuberculosis (TB*), M. avium (AV*), M. szulgai (SZ*), M. kansasii (KA*), M. xenopi (XE*) and M. simiae (SI*) were hybridized on the nylon membrane. Specific differential hybridization is obtained.

FIG. 7. Example of biochips detecting specifically M. gordonae

FIG. 8. Alignment of several Mycobacterial us-p34 sequences.

FIG. 9. Alignment of three Mycobacterial us-p34 sequences (M. tuberculosis, M. avium and M. intracellulare.

FIG. 10. Alignment of several pairs of Mycobacterial us-p34 sequences. FIG. 11.

A. Organisation of the rrn operon of Pseudomonas aeruginosa (Ps.ae), Burkholderia cepacia (Bu.ce) and Stenotrophomonas maltophilia (St.ma).

B. Comparison of the sequences flanking the regions encoding 16S and 23S RNA from Pseudomonas aeruginosa (Ps.ae) (SEQ ID NO 76), Burkholderia cepacia (Bu.ce) (SEQ ID NO 77). CNS: consensus.

FIG. 12. Discrimination by multiplex PCR.

The DNA from Ps. aeruginosa (1), Bu. cepacia (2) or St. maftophilia (3) was amplified using a conserved primer and a set of specific primers.

A. Multiplex PCR theoretical scheme.

B. Agarose Gel allowing the visualization of the amplified fragments of different sizes. In a, molecular weight marker (scale 123 bp).

FIG. 13. Multiplex PCR sensibility validation

The DNA from clinical strains (1 to 10) and from Ps. aeruginosa (A), Bu. cepacia (B) or St. maltophilia (C) were amplified using PsMulti, Paer, Bcep and Smal primers. The amplified fragments (20 μl) are visualized after agarose gel (2 %) migration.

In a, molecular weight marker (scale 123 bp). In b, c and d are control fragments amplified from Ps. aeruginosa, Bu. cepacia and St. maltophilia respectively.

FIG. 14.

Multiplex PCR specificity validation

In a, molecular weight marker (scale 123 bp). In b, c and d are control fragments amplified from Ps. aeruginosa, Bu. cepacia and St. maltophilia respectively.

The DNA from clinical strains of Ps. putida (1), Ps. fluorescens (2) or Ps. alcaligenes (3), Ps. pseudoalcaligenes (4) and Ps. stuzeri (5) were amplified using PsMulti, Paer, Bcep and Smal primers. The absence of amplification products (20 μl) was visualized after agarose gel (2%) migration.

Inset: The DNA from clinical strains of Ps. putida (1), Ps. fluorescens (2) or Ps. alcaligenes (3), Ps. pseudoalcaligenes (4) and Ps. stuzeri (5) was amplified using ProUni1 and ProUni2 primers (universal primers amplifying part of the 16S RNA gene). The amplification products (20 μl) were visualized after agarose gel (2%) migration.

FIG. 15. Reverse hybridization for the discrimination between Ps. aeruginosa, Bu. cepacia and St. maltophilia

2 and 5 μl of probes corresponding to the three species where sequentially immobilized on Hybond N+nylon membrane (from top to bottow, 2 μl and 5 μl of the probe specific for Ps. aeruginosa, 2 μl and 5 μl of the probe corresponding to Bu. cepacia and 2 μl and 5 μl of probe specific for St. maltophilia). Biotinylated amplicons were produced from the genomic DNA of each species and were hybridized with a membrane containing the different probes. The hybrids were calorimetrically detected.

FIG. 16.

Visualization of the second rrn operon from Ps. putida

A. Amplification products (20 μl) from DNA of Ps. aeruginosa (1), Bu. cepacia (2), St. maltophilia (3), Ps. putida (4), Ps. alcaligenes (5), Ps. pseudoalcaligenes (6), Ps. fluorescens (7) using PsSeql and PsSeq2 primers, visualized on agarose gel (2%).

B. The DNA was transferred from the gel A to a nylon membrane, and hybridized with a biotiylated probe (DNA from Ps. aeruginosa amplified with PsSeql and psSep2b primers). The hybrids were revealed by colorimetry (streptavidine, DAB). PM=molecular weight marker

FIG. 17.

Alignment of the two rrn operon sequences from Ps. putida. First (top) sequence is SEQ ID NO 78, second (bottom) sequence is SEQ ID NO 79.

The sequence of the shortest operon is boxed, the double-underlined sequence correspond to the missing region of the shortest operon.

FIG. 18.

Alignment and consensus sequence between Pseudomonas aeruginosa (ps.msf{padfc}), Burkholderia cepacia (ps.msf{pcdfg} and Stenotrophomonas maltophilia (ps.msf{xmdfa}). The underlined sequences correspond to the regions where the primers for amplification were chosen.

FIG. 19.

Alignment without consensus sequence between Pseudomonas aeruginosa (ps.msf{padfc}) (SEQ ID NO 80), Burkholderia cepacia (ps.msf{pcdfg} (SEQ ID NO 81) and Stenotrophomonas maltophilia (ps.msf{xmdfa}) (SEQ ID NO 82).

### EXAMPLES

### Example 1

### Differential diagnosis of Mycobacterial species

1. Preparation of mycobacterial DNA.

a) short protocol (mycobacteria obtained from the Pasteur Institute): DNA has been obtained by boiling the samples in order to put the mycobacterial DNA into solution.

b) long protocol (mycobacteria obtained from the Institute of Tropical Medicine): Mycobacteria (10 mg [wet weight]) were suspended in 200 μl of lysis solution (0.1 M NaOH, 1 M NaCl, and 5% sodium dodecyl sulfate [SDS]) and heated (100° C.) for 20 min. The suspension was then cooled, neutralized with 3 volumes of 0.1 M Tris-HCl (pH 7.4) buffer, and centrifuged (5,000 ×g, 5 min). Supernatants were extracted with phenol-chloroform, and DNA was precipitated with ethanol, collected by centrifugation, dissolved in 50 μl of H₂O, and stored at 20° C.

2. PCR amplifications.

For amplification, an aliquot (10 μl ) of the DNA samples was added to 90 μl of PCR mixture consisting of 10 mM Tris-HCl (pH 8.8), 1.5 mM MgCl₂, 50 mM KCl, 0.1% Triton X-100, 0.25 mM (each) deoxynucleoside triphosphates, 10 pmol of each primer (Tables 1 and 2) and 0.625 U of DyNAzyme DNA polymerase (Finnzymes Inc., Espoo, Finland). After an initial denaturation step (3 min at 96° C.), 30 cycles of amplification were performed as follows: denaturation at 96° C. for 30 s, annealing at 58° C. for 45 s, and DNA extension at 72° C. for 30 s, with an increment of 1 s per cycle for the denaturation and extension segments. A final extension was performed at 72° C. for 15 min. Amplifications were carried out in a DNA 2400 thermocycler (Perkin-Elmer Applied Biosystems, Foster City, Calif.). PCR products were checked by loading 5 μl on a 2% (wt/vol) agarose gel. Electrophoresis is performed in 0.1 M Tris HCl (pH 8.6), 80 mM boric acid, 1 mM EDTA buffer containing 0.5 μg of ethidium bromide per ml. DNA fragments are visualized on a UV transilluminator at 300 nm. PCR products-were cloned using the TOPO XL PCR cloning kit (Invitrogen, Carlsbad, Calif.), according to the manufacturer's protocol. The clones were further sequenced with the Taq Dye Deoxy Terminator Cycle sequencing kit and an ABI 377 DNA sequencer (Perkin-Elmer Applied Biosystems).

3. DNA sequence analysis.

Nucleotide sequence analyses were performed with the Genetics Computer Group software obtained from the University of Wisconsin, through the use of the Belgian EMBnet Node facility. Sequences were aligned by the Pileup program and paired comparisons were realised with the Bestfit program. 4. Reverse hybridization analysis. Mycobacterial species-specific capture probes were produced by amplification of the us-P34 cloned-DNA from the reference strains with primers U1 and U4. Amplified DNA fragments were sequentially transferred onto nylon membranes (Hybond-N+; Amersham, Little Chalfont, United Kingdom) according to the Southern blot method. After 2 h of prehybridization, membranes were hybridized with us-p34 digoxigenin-labeled target probes, obtained by PCR amplification of reference or clinical mycobacterial genomic DNA with primers U1 and U4 (Table 1), in the presence of DIG-1 1 -dUTP. Hybridization of heat denatured target probes (5 min at 95 ° C.) was performed at 50 ° C. for 4 h in 2x SSC (lx SSC is 0.15M NaCl and 0.015 M sodium citrate), 1% blocking reagent, 0.1% SDS, 0.1% N-Lauroylsarcosine, 5 mg/ml of salmon sperm DNA and 5% formamide. Filters were then washed twice with 2x SSC (lx SSC is 0.15 M NaCl plus 0.015 M sodium citrate)-0.1% SDS at 37° C for 5 min, and twice with 0.2x SSC-0.1% SDS at 50° C. for 5 min. Hybridized digoxigenin-labeled DNA fragments were detected through alkaline-phosphatase-labeled anti-DIG Fab fragments. Colorimetric detection was performed with Nitro Blue Tetrazolium Chlorure and 5,3-bromo-4-indolylphosphate (BCIP) (Boerhinger Mannheim) according to the manufacturer's instructions.

5. Analysis on “Mycobacteria Biochips”.

Design of the biochips. Mycobacterium micro-arrays have been developed by AAT (Namur, Belgium). This array is a glass slide bearing on its surface different mycobacterial species-specific capture nucleotide sequences (Table 3). Fixation, positive and negative hybridization controls capture probes are also included in the array and each capture probe of the array is composed of four replicates.

Production of biotinylated amplicons. Amplification of the mycobacterial us-p34 sequences is performed in a final volume of 50 μl containing 2.5 mM MgCl₂, 75 mM Tris-HCl, pH 9.0, 50 mM KCl, 20 mM (NH₄)₂SO₄, 0.5 μM of primers U4 and U5, 200 μM of dATP, 200 μM of dCTP, 200 μM of dGTP, 150 μM of dTTP, 50 μM of biotin-16-dUTP, 0.5 U of uracil-DNA-Glycosylase (Boehringer Mannheim, Germany), 1.25 U of Taq DNA polymerase (Biotools, Madrid, Spain) and 10 μl of DNA template. The reagents are first incubated at 94° C. for 5 min and then cycled 40 times in a DNA 9600 thermocycler (Perkin Elmer, Foster City, Calif., USA) using the following temperatures and cycle times: 94° C. for 30 s, 49° C. for 45 s, 72° C. for 30 s. A final extension step of 10 min at 72° C. is performed. PCR products are directly used or stored at −20° C.

Hybridization and calorimetric detection. The procedure for detecting the PCR products is carried out according to the manufacturer's instructions (AAT, Namur, Belgium) as follows: 5 μl of PCR product and 5 μl of the positive control, provided in the kit, are denatured with fresh NaOH 0.05N for 5 min at room temperature. This solution is then mixed with 35 μl of hybridization solution and loaded on the array framed by a hybridization chamber (Biozym, Landgraaf, The Netherlands). The chamber is closed with a plastic coverslip and hybridization is carried out for 30 min at 53° C. Slides are washed four times 1 min with washing buffer. The glass samples are then incubated 45 min at room temperature with 800 μl of streptavidin-conjugate 1000 times diluted in blocking buffer. After 5 washes, the slides are incubated 3 times 10 min with 800 μl of revelation mixture, then rinsed with water, dried and imaged using a calorimetric microarray reader (AAT, Namur, Belgium). While a quick visual inspection already yields most of the images relevant informations, the image obtained is analysed by an algorythm allowing the quantification of the spots intensities values and by a pattern recognition algorithm.

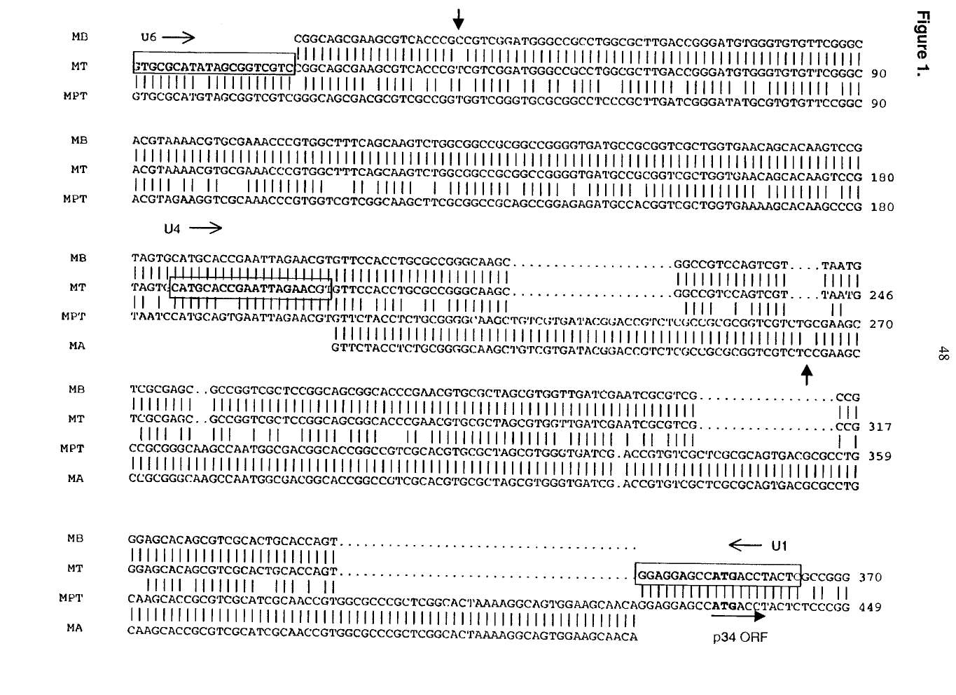

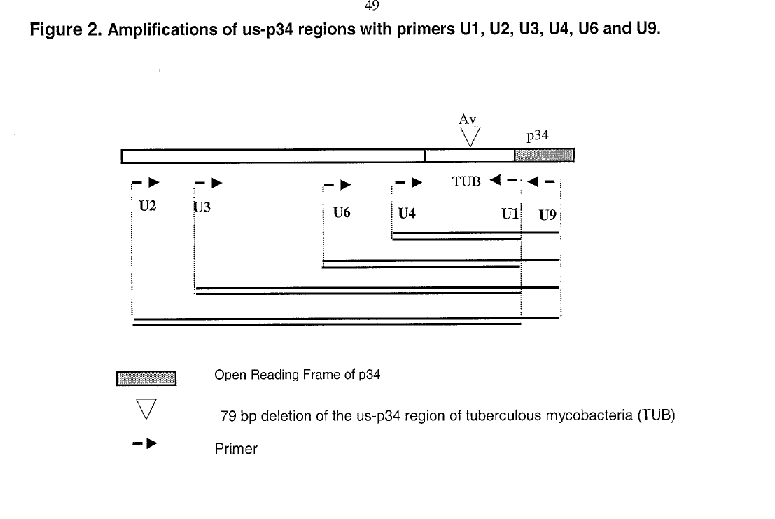

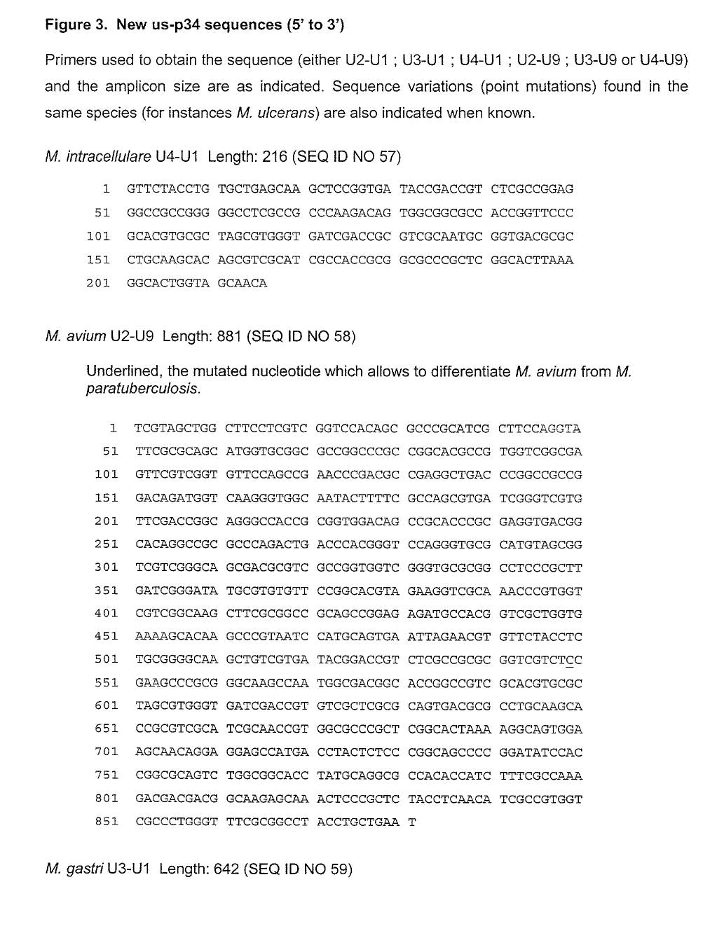

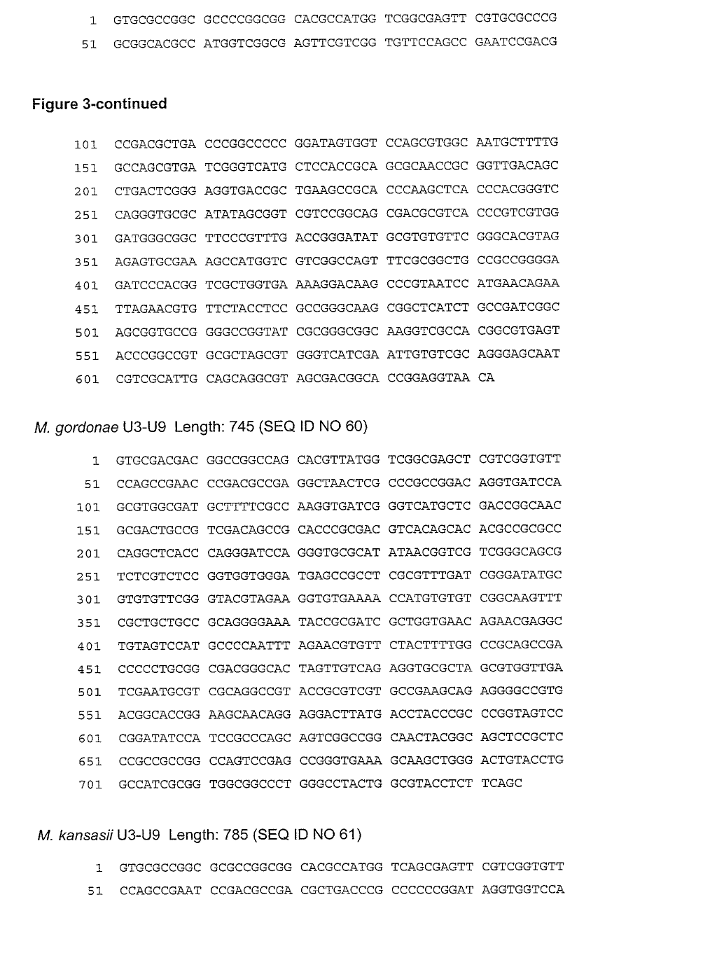

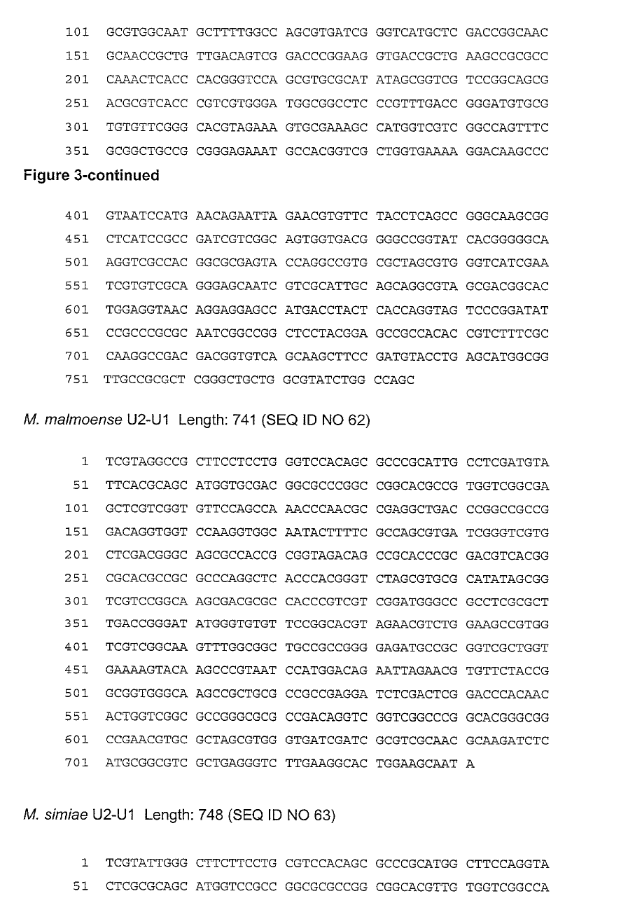

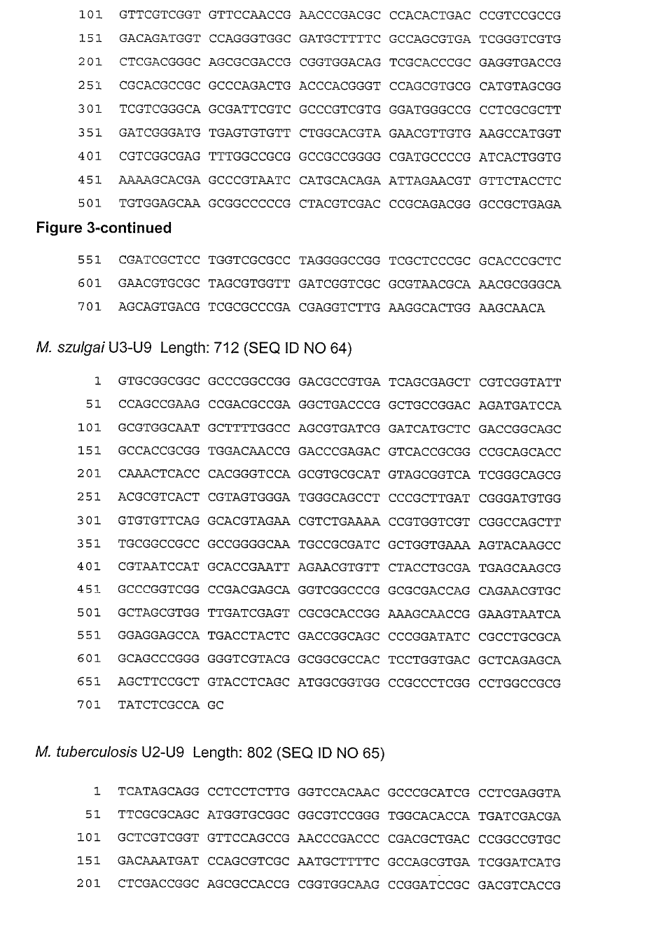

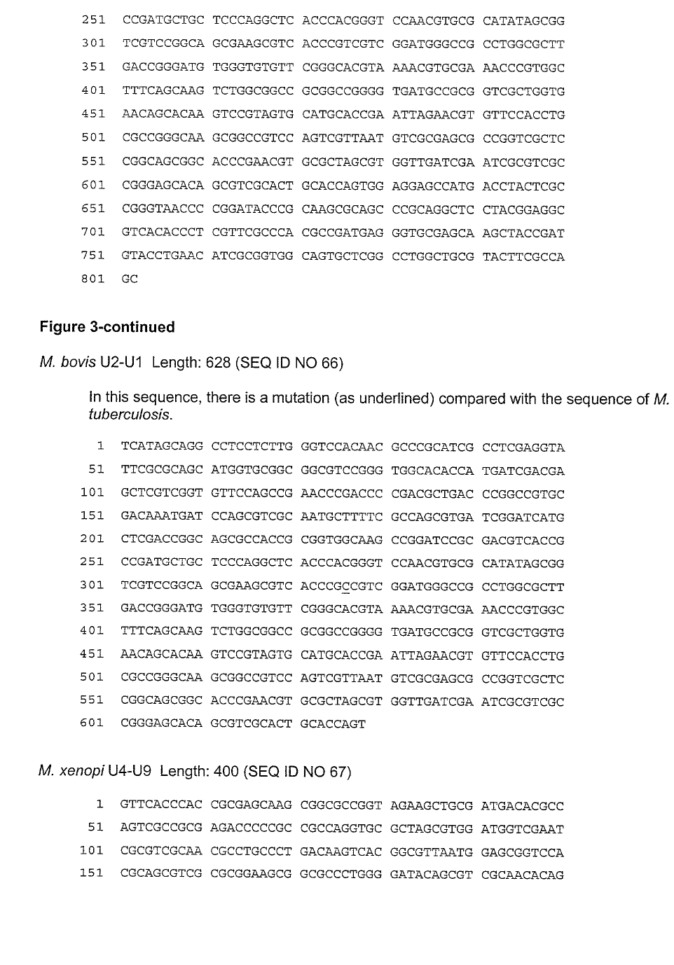

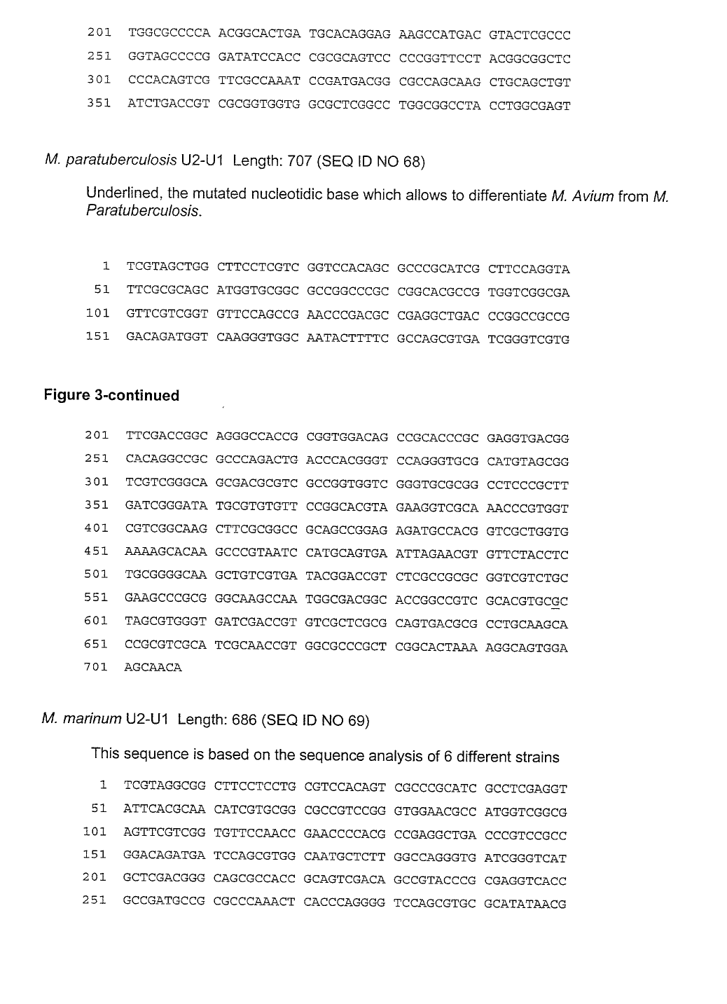

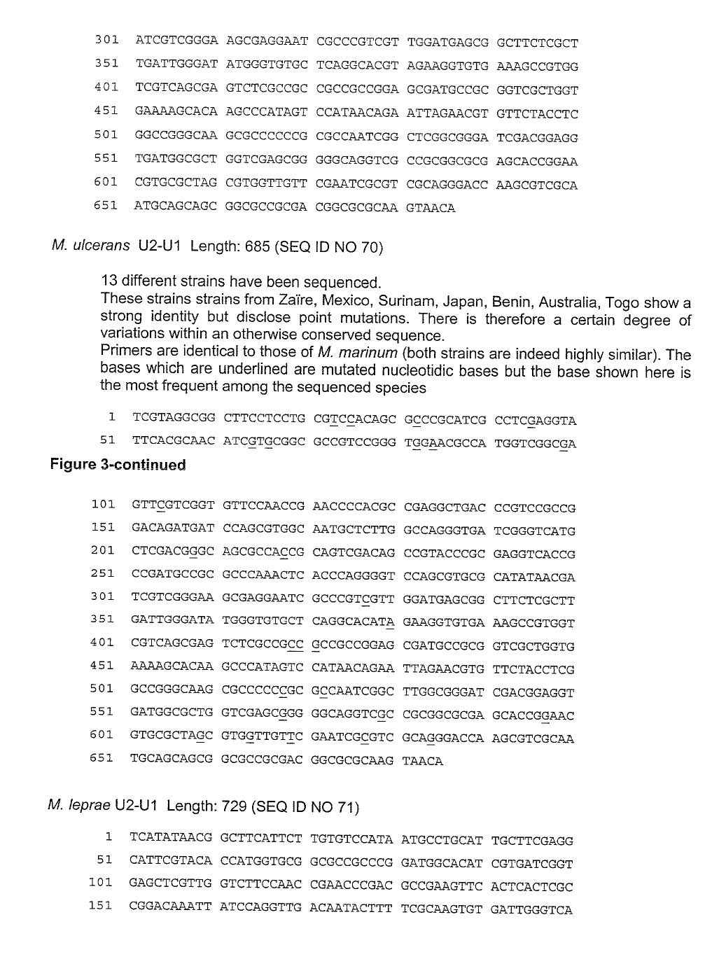

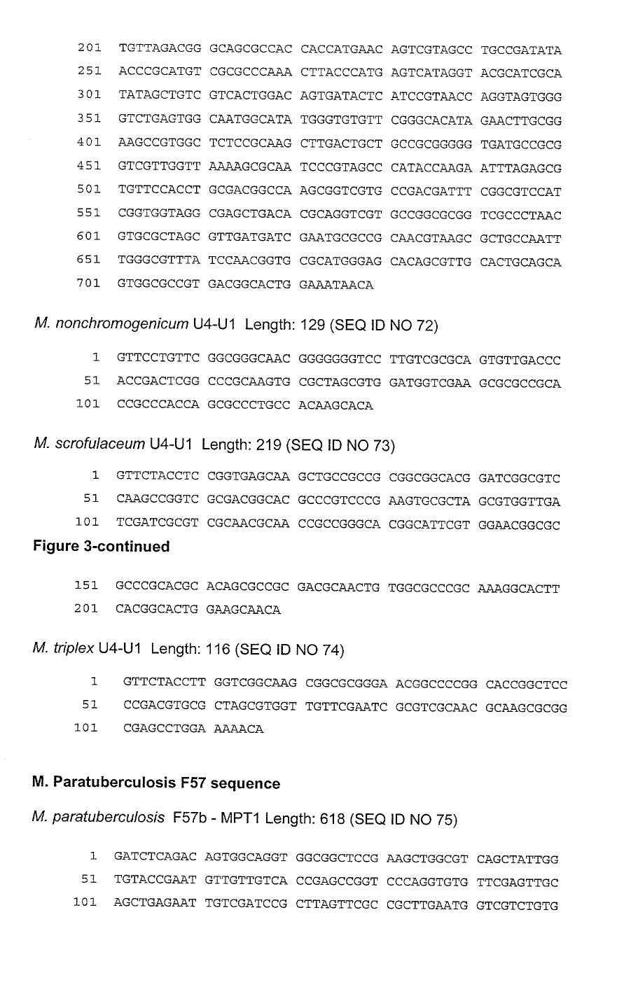

6. Cloning and sequencing of the upstream-P34 region of mycobacterial species. Comparison of homologous p34 genes and its regulatory sequences (upstream p34 or us-p34) from M. tuberculosis (GenBank accession no. Z79700) and M. avium subsp. paratuberculosis (GenBank accession no. X68102) showed the presence of deletions in the M. tuberculosis region upstream of the p34 protein start codon (FIGS. 1 and 2). To confirm and extend these findings, the us-p34 sequence of M. bovis BCG and M. avium D4 were amplified and sequenced. Multiple sequences alignment of this region revealed interspecies polymorphisms specific for both tuberculous (M. tuberculosis and M. bovis Bacile de Calmette et Guérin [BCG]) and non-tuberculous (M. avium sp and M. avium subsp. paratuberculosis) mycobacteria, i.e. the us-p34 fragment was 79 bases shorter in the tuberculous species. Conversely, us-p34 appeared to be highly conserved within each group: differentiation between M. tuberculosis and M. bovis relied on a single T to C transition at position 41 of us-p34, and a single C to G transversion at position 264 of us-p34 in M. avium ss and M. avium subsp. paratuberculosis (FIG. 1). Based on sequence homology between the M. tuberculosis/M. bovis and M. avium/M. avium subsp. paratuberculosis, several oligonucleotides, U1, U2, U3, U4 and U9 (Tables 1 and 2), matching conserved sequences of the polymorphic us-p34 region, were designed to amplify the corresponding region of others reference bacteria from the mycobacterial genus, M. szulgai, M. africanum, M. gordonae, M. gastri, M. kansasii, M. marinum, M. ulcerans, M. intracellulare, M. scrofulaceum, M. xenopi, M. malmoense and M. simiae (Table 3). These fragments have been cloned and sequenced (FIG. 3).

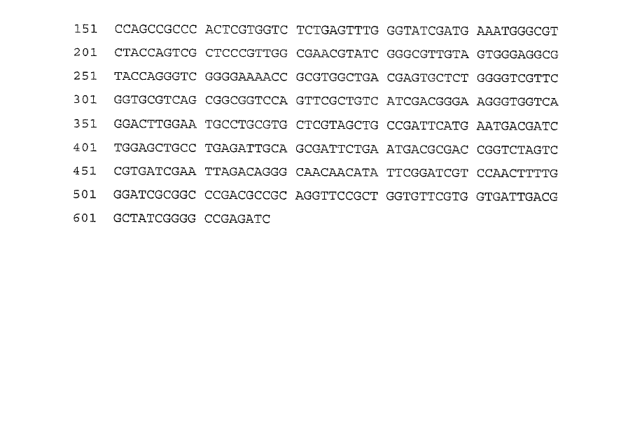

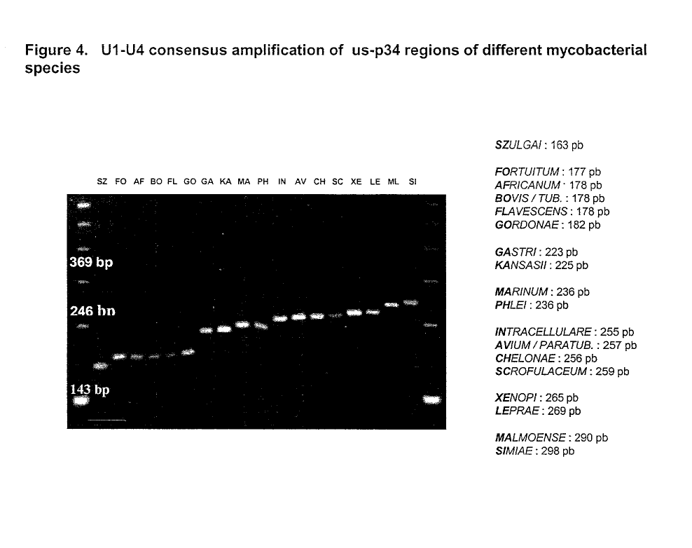

7. Development of a consensus PCR amplication of mycobacterial species. Based on the mycobacterial sequences of us-p34, a consensus PCR assay was developed. Primers U1 and U4 have allowed to amplify the corresponding fragments, ranging from 163 bp and 298 bp in size, for M. szulgai (163 bp), M. non chromogenicum (169 bp), M. tuberculosis, M. bovis, M. africanum (178 pb), M. gordonae (182 bp), M. gastri (223 bp), M. kansasii (225 bp), M. marinum, and M. ulcerans (236 pb), M. intracellulare, M. avium, M. paratuberculosis (256 bp), M. scrofulaceum (259 bp), M. xenopi (265 bp), M. leprae (269 bp), M. malmoense (290 bp) and M. simiae (298 bp) (FIG. 4; Table 4). Sequences alignments indicate both size and nucleotide sequence polymorphisms, as illustrated by some pair-wised alignments of all mycobacterial sequences with M. tuberculosis us-p34 region.

8. Validation of the consensus mvcU1-mvcU4 PCR on clinical isolates. The reliability of this consensus amplification strategy was tested on a range of clinical mycobacterial isolates (Table 4). The strategy is characterized by a good sensitivity for most mycobacteria. The lower sensitivity displayed for some species is probably related rather to the DNA sample than to a lack of sensitivity of the strategy. As a matter of fact, M. marinum-M. ulcerans amplifications show a better sensitivity for “long protocol” DNA preparations (20/20) than for the “short protocol” ones (4/10). The quality of these “short protocol” DNA preparations have to be checked before running further conclusions. This could indeed influence further identification of potentially specific amplicons in other mycobacterial species (such as, for example, M. phlei, M. flavescens, M. nonchromogenicum, M. chelonae)

9. Development of species-specific PCR amplifications.

Based on alignment of all upstream p34 sequences between U1 and U4 (which contain most inter-species differences) or at the broadest between U9 and U2 (which also may contain specific inter-strain differences), primers which will selectively amplify one strain (two new primers or a combination of one new primer and one primer presented in Tables 1 and 2) are designed. This strategy enables the differentiation of the mycobacterial species by PCR.

10. Reverse hybridization strategy.

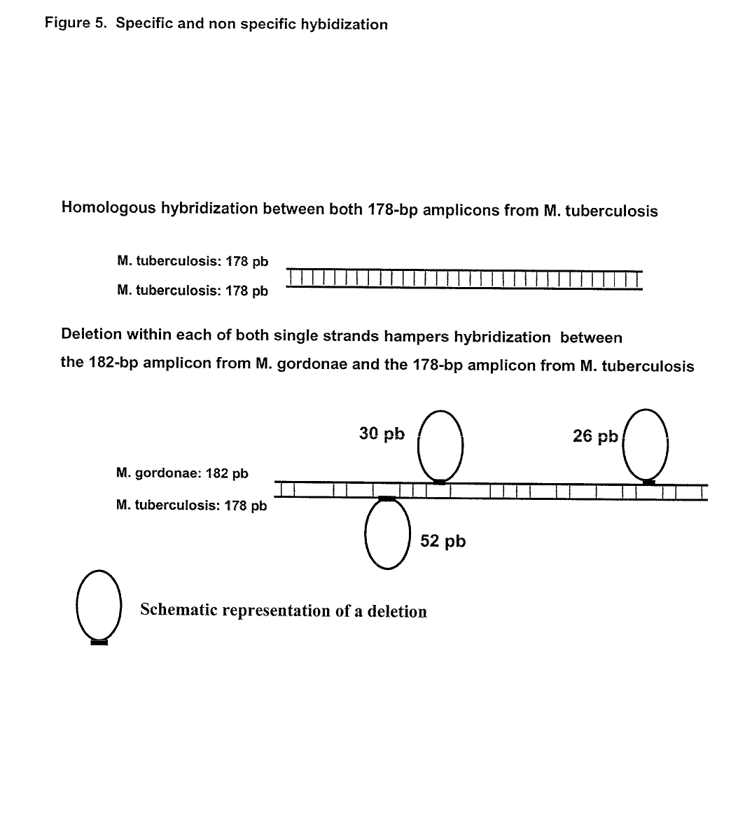

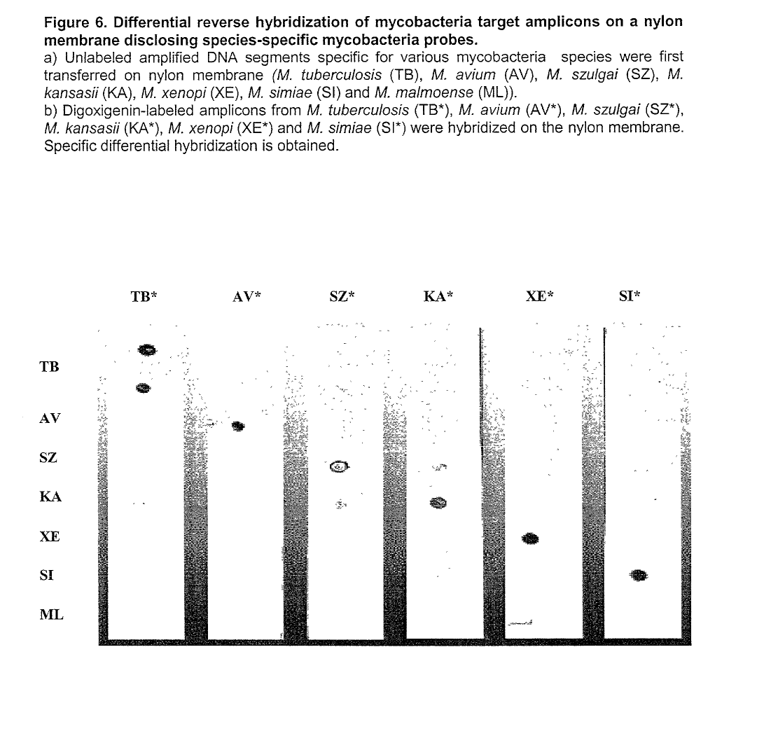

Based on homologous and non-homologous hybridization of amplicons from the same species and amplicons from two different species, respectively (FIG. 5), a reverse hybridization assay was set up. In a first step, species-specific probes corresponding to each mycobacteria species, were produced by amplification of reference strains us-p34 cloned DNA with the two consensus primers U1 and U4 and were immobilized on nylon strips. DNA of species to be identified were amplified in the presence of the same primers, biotinylated at their 5′ ends, hybridized with the immobilized probes and calorimetrically assayed. An assay, allowing a correct discrimination between M. tuberculosis, M. avium, M. szulgai, M. xenopi, M. simiae and M. malmoense is presented in FIG. 6.

11. Development of a “Mvcobacteria Biochips'.

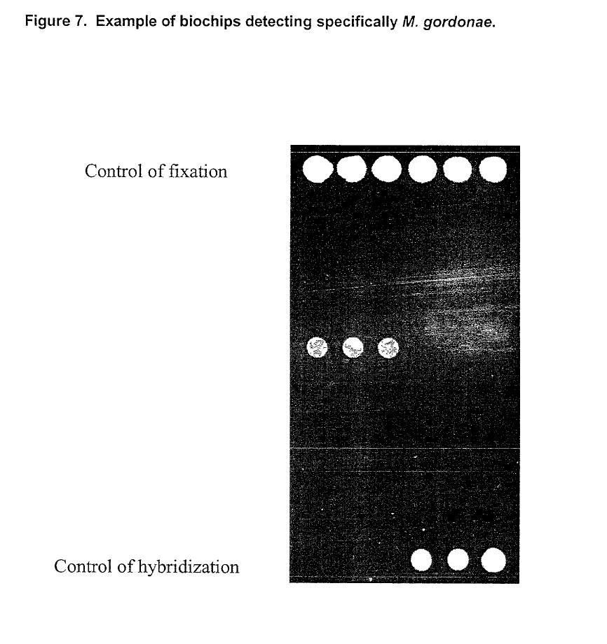

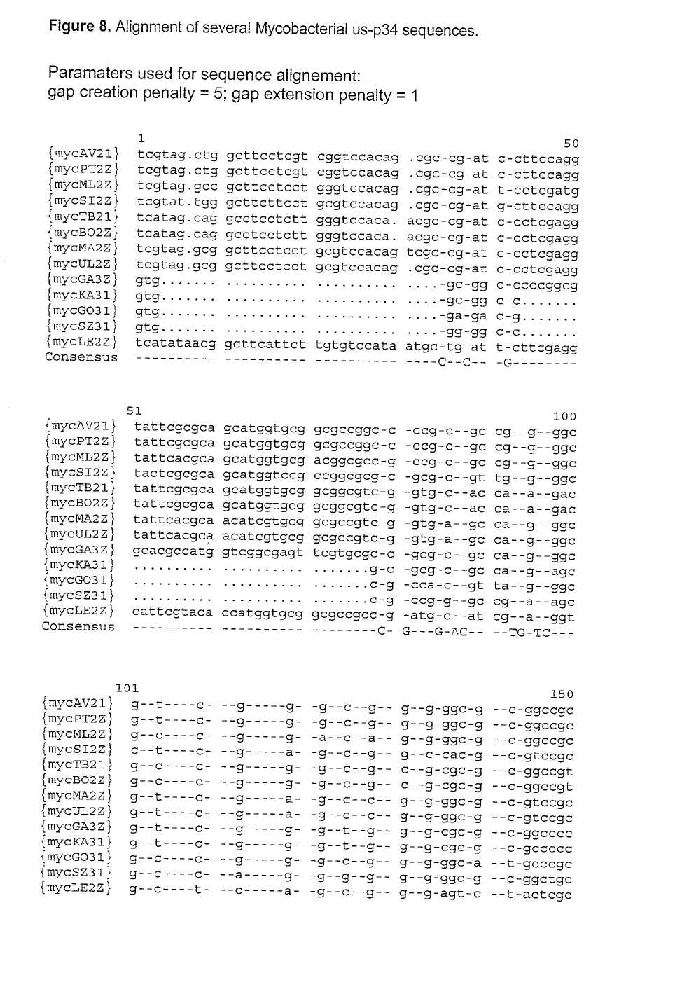

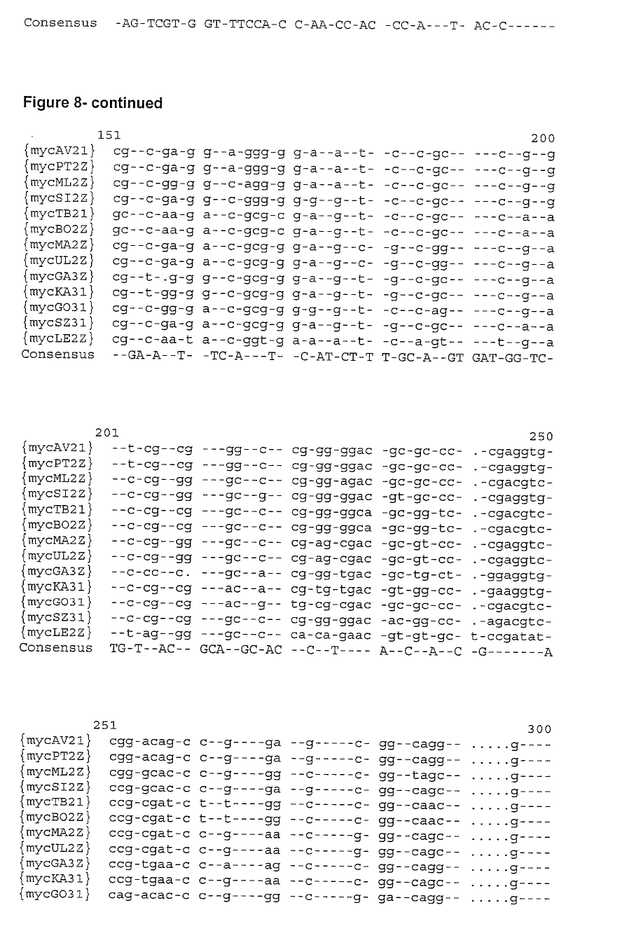

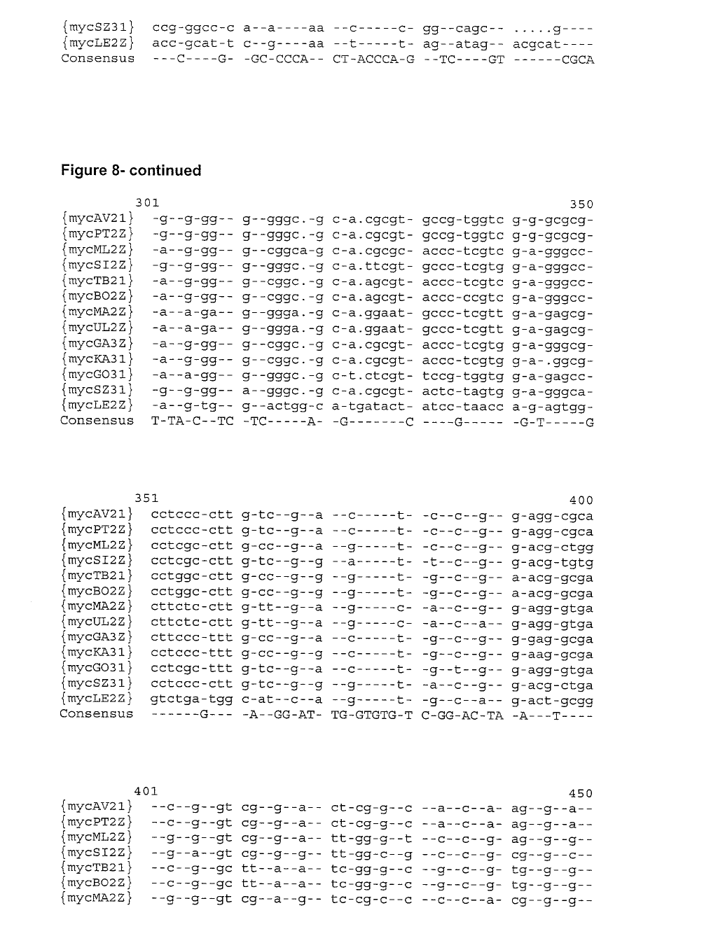

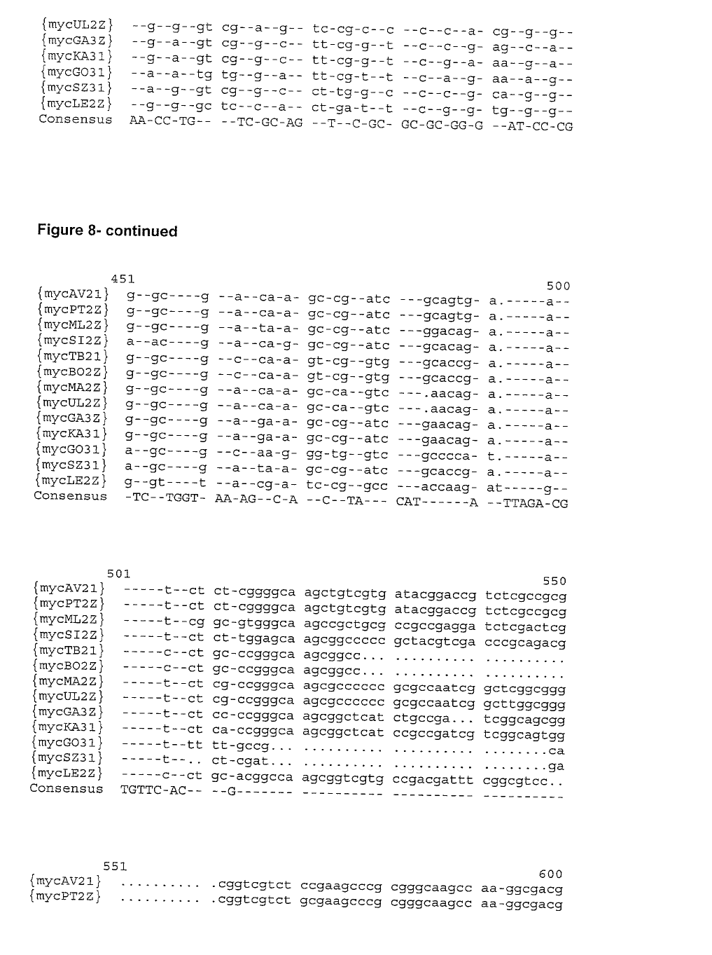

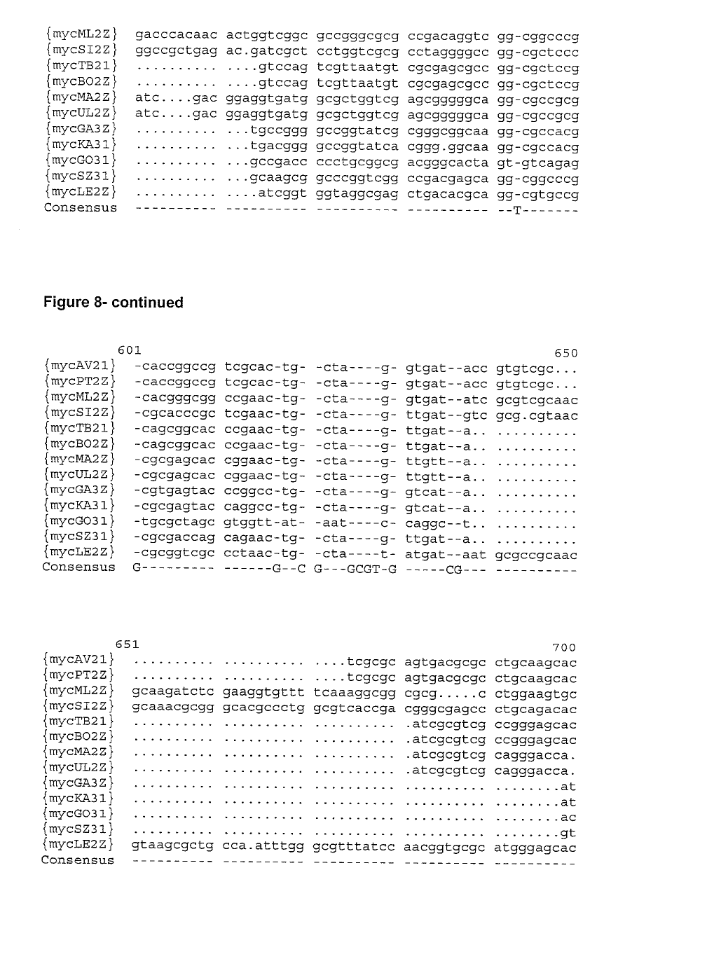

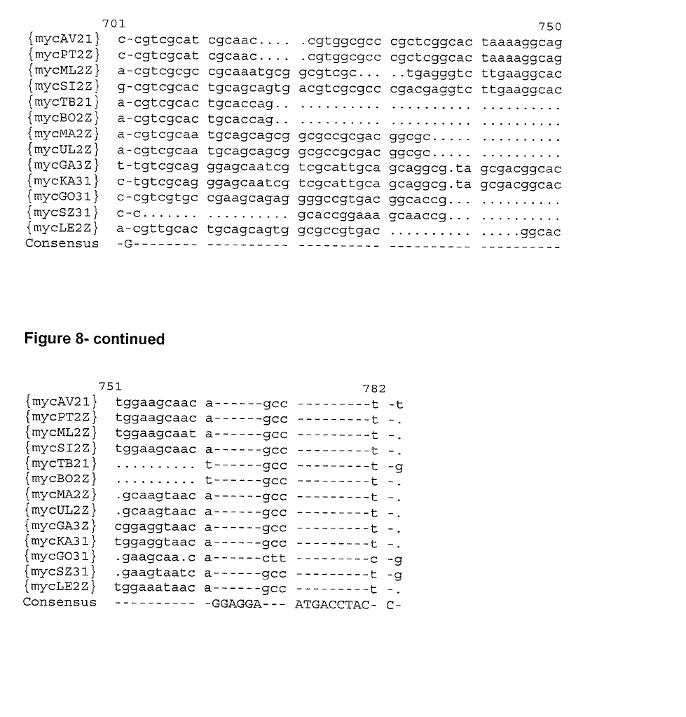

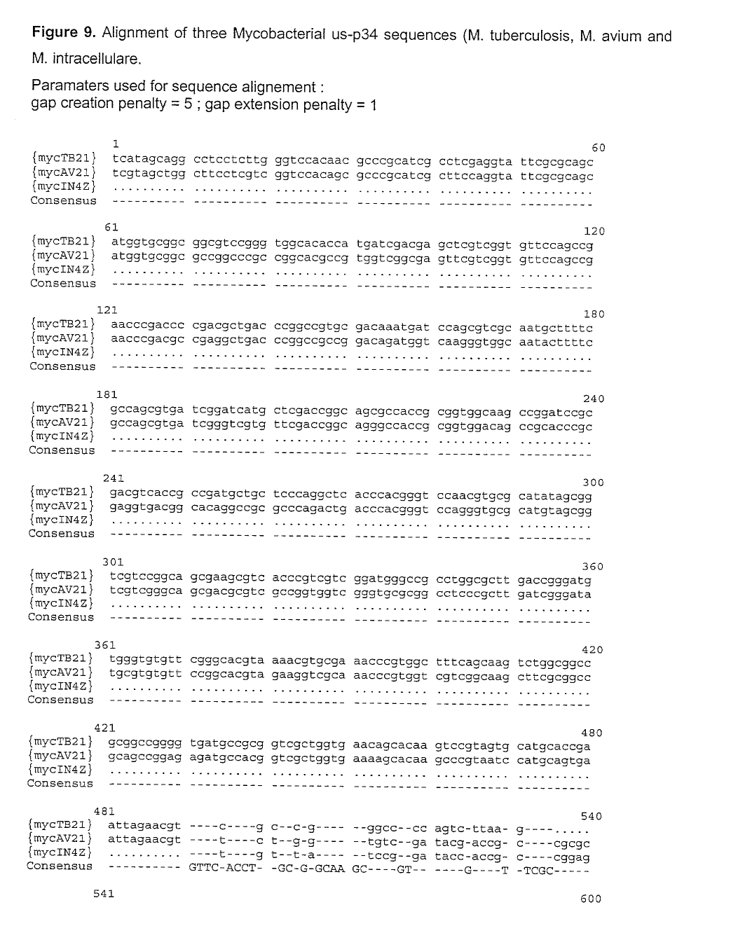

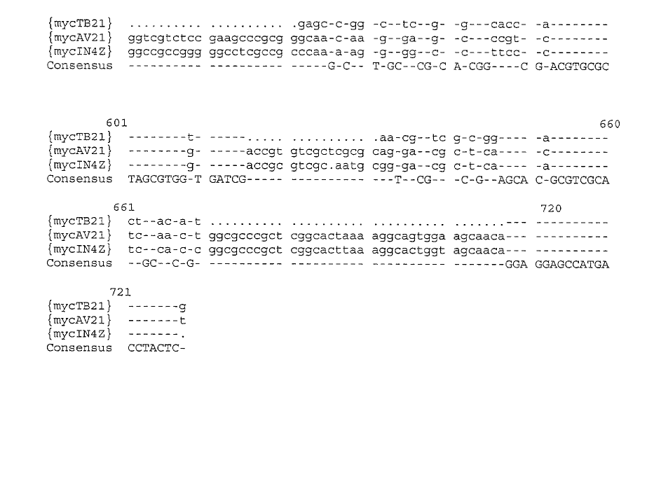

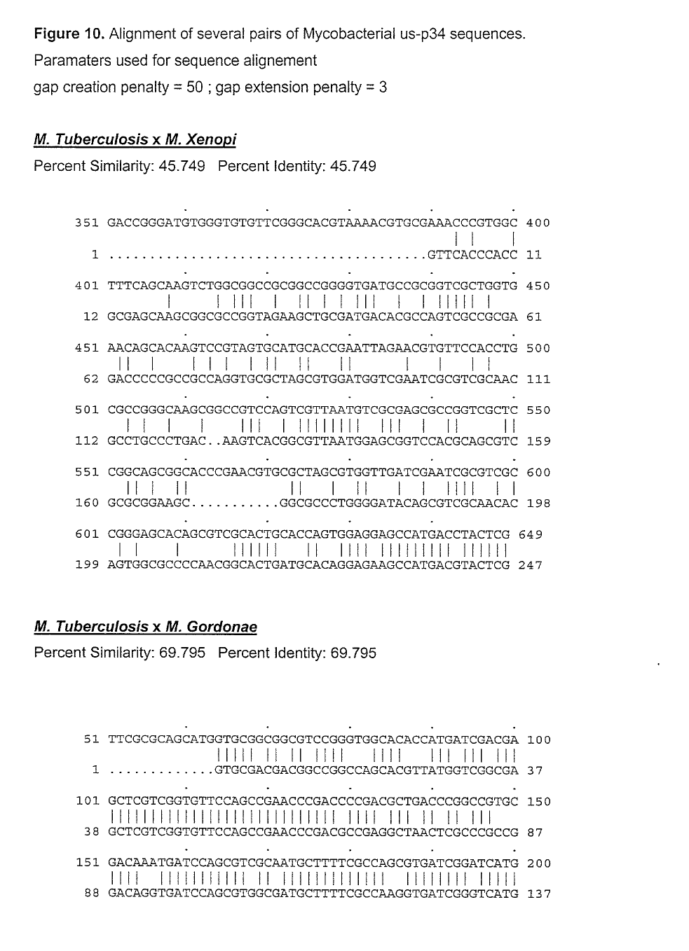

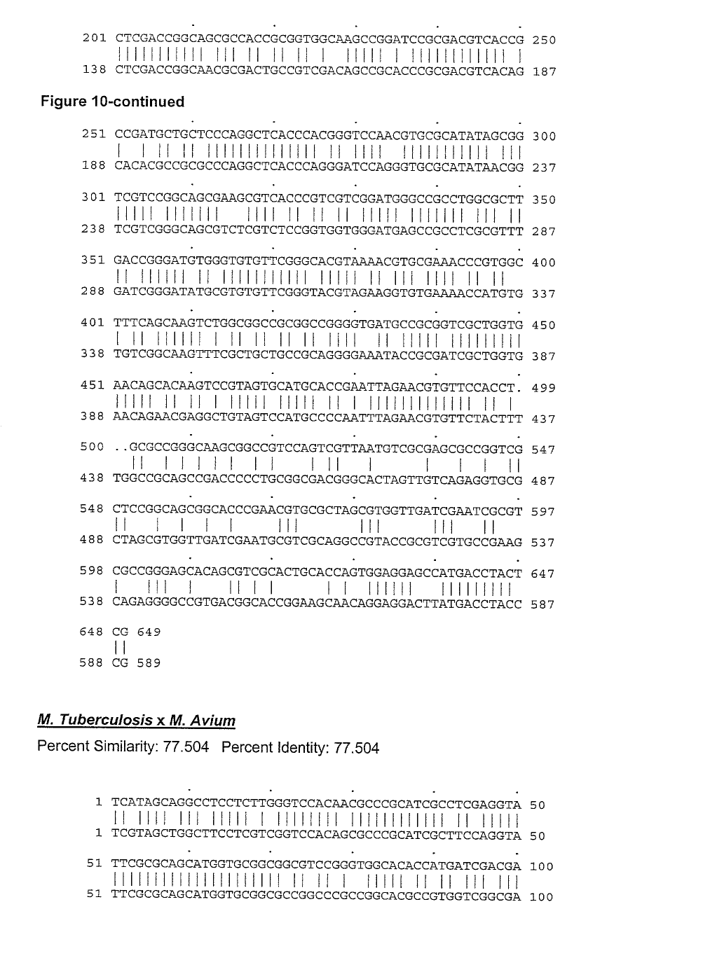

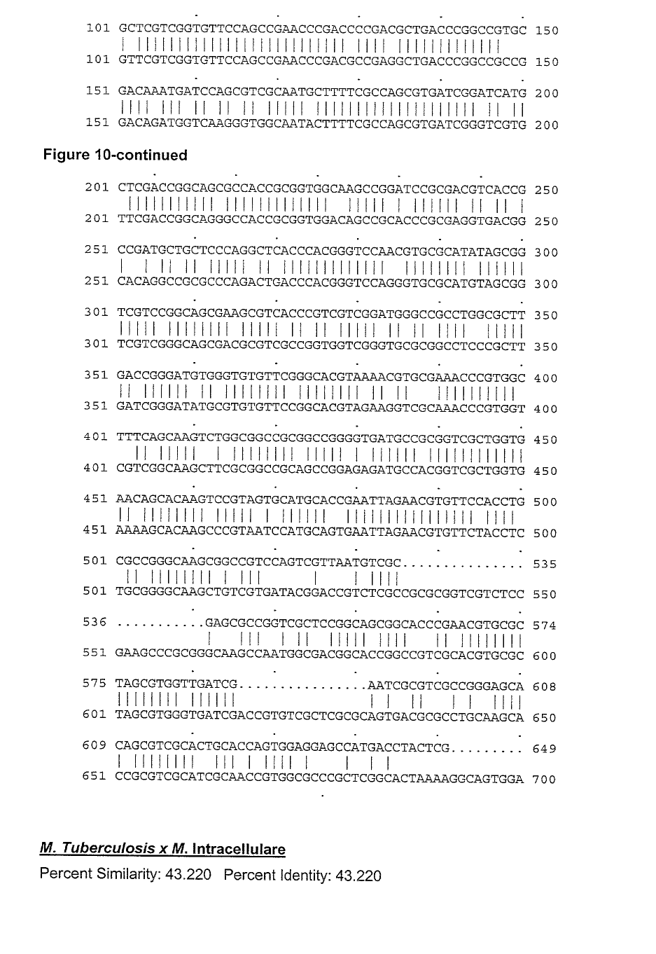

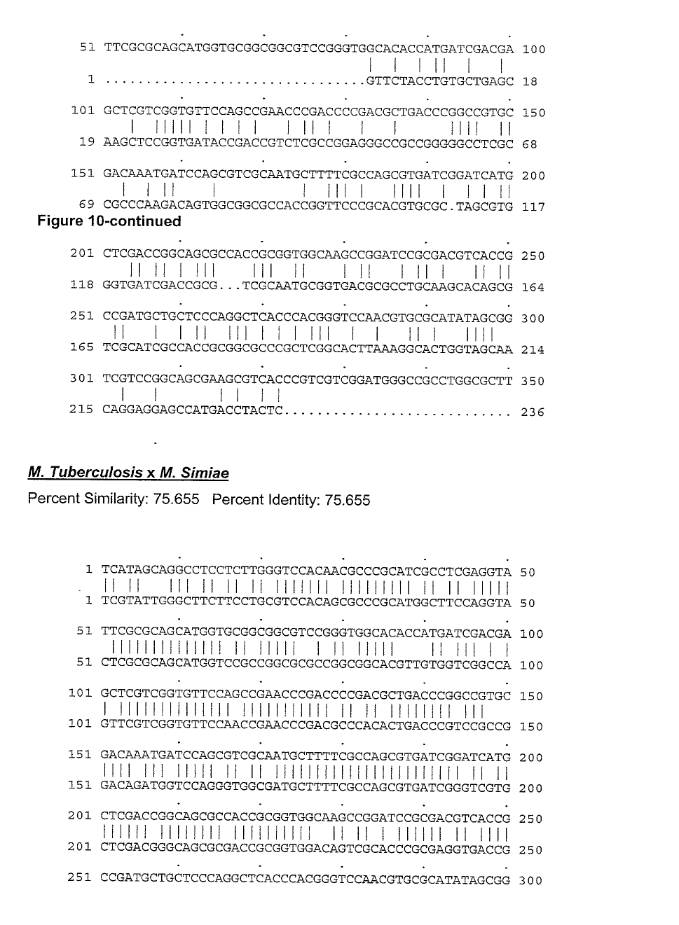

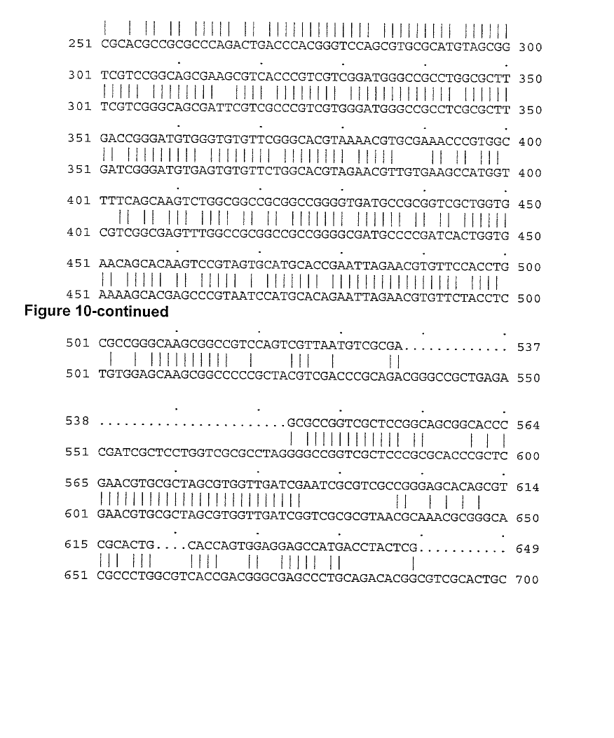

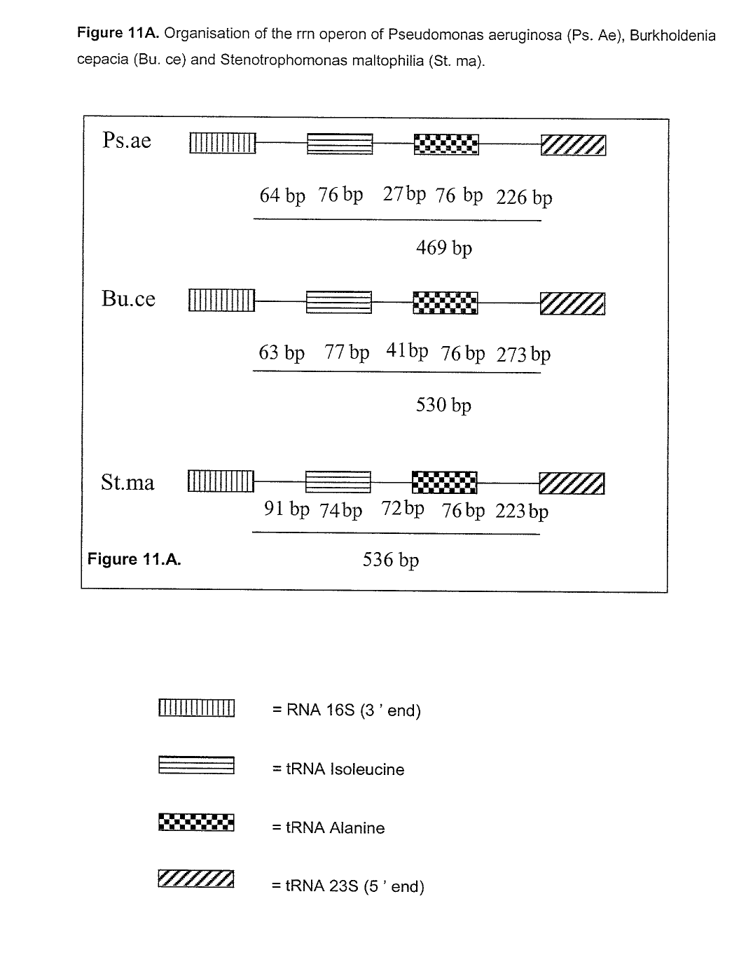

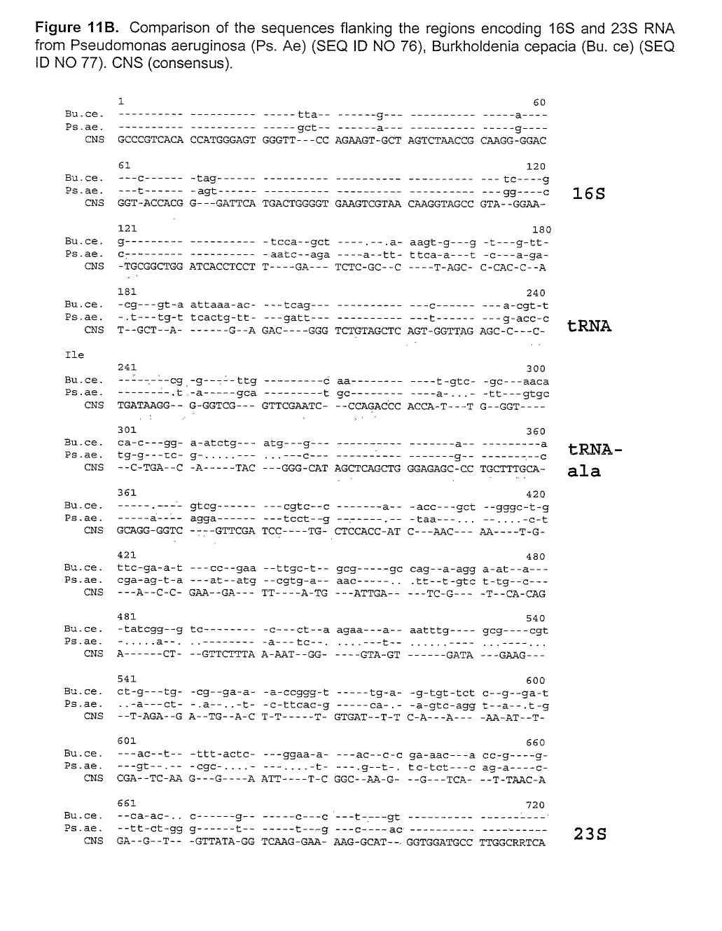

The polymorphic us-p34 sequences can also be applied on low-density microarray technology in order to develop simultaneous species-specific detection of mycobacteria. In a preliminary study, mycobacteria microarrays, bearing different mycobacterial species-specific capture probes, have been developed by AAT (Namur, Belgium). These probes were hybridized with the us-p34 biotinylated amplicons from the different mycobacterial species. Illustration of a specific detection of M. gordonae versus M. tuberculosis, M. gastri, M.kansasii, M. intracellulare, M. leprae, M. avium, M. marinum, M. simiae, M. xenopi, M. malmoense, M. szulgai and M. scrofulaceum specific capture probes is presented in FIG. 7.

### Example 2

### Differential diagnosis of Pseudomonas species

1. The Pseudomonas

The Pseudomonas belong to the family of non-fermenting bacteria (7, 8). They are divided into 5 groups based on the homology level of their ribosomal RNA (Table 5). From a clinical point of view, the three species most frequently found are: Pseudomonas aeruginosa, Burkholderia cepacia, Stenotrophomonas maltophilia.

2. Differential diagnosis

The present inventors developed a method of differential identification allowing the discrimination of three species belonging to the same family of Pseudomonads: Pseudomonas aeruginosa, Burkholderia cepacia, Stenotrophomonas maltophilia. The identification method is based on the rRNA operon (rrn), and more particularly on the variable region (spacer) which separates the different genes coding for 16S and 23S rRNA and the Isoleucine and Alanine tRNA.

Some studies have been published related to the amplification of the gene coding for 16S and 23S ribosomal RNA (ref 7 to 9, non-exhaustive).

3. Organization of the rrn operon of “Pseudomonas”

The bacteria belonging to the “Pseudomonas” group have an operon coding for the three ribosomal RNA, 16S, 23S and 5S. The organization of this operon is relatively conserved (FIG. 11A). Between the sequences encoding 16S and 23S rRNA, one finds sequences coding for two tRNA (Isoleucine and Alanine tRNA) separated by non-coding regions of variable length and composition (FIG. 11A and 11B). It is also noticed that these two tRNA have the following order in P. aeruginosa and B. cepacia: Isoleucine tRNA followed by Alanine tRNA and that this order is reversed in S. maltophilia.

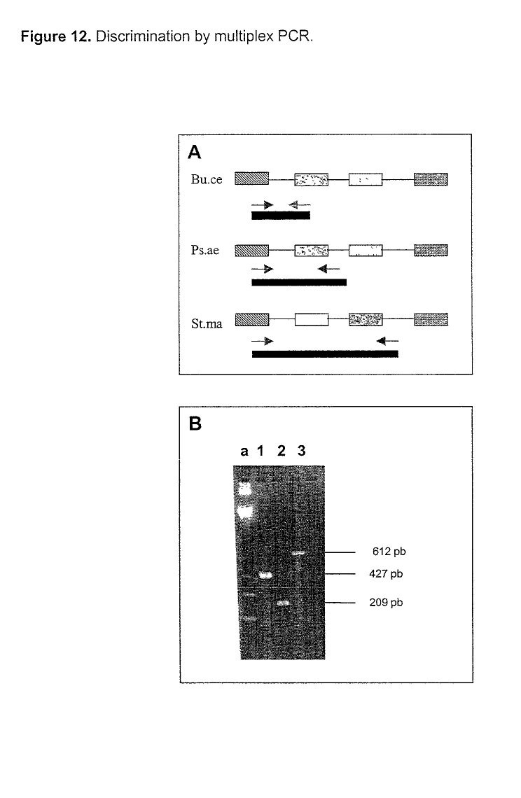

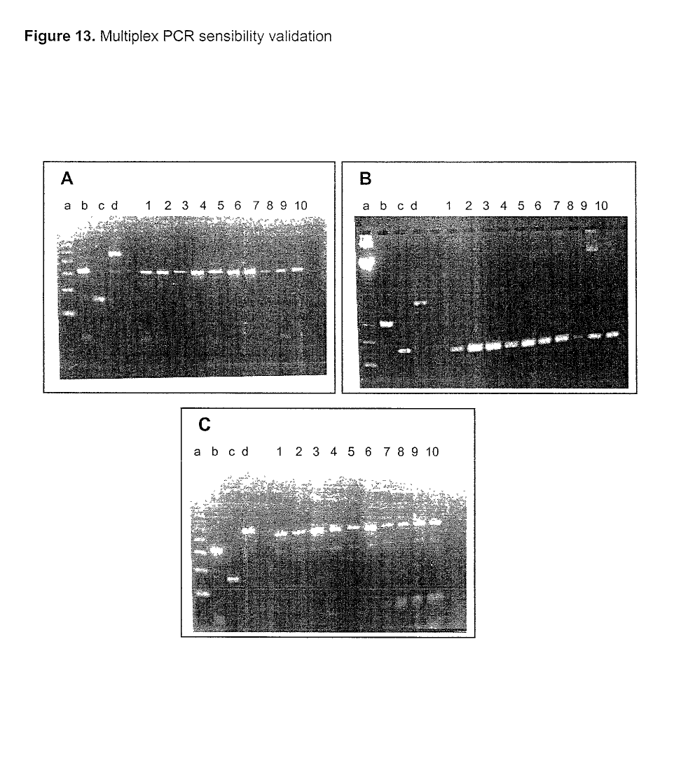

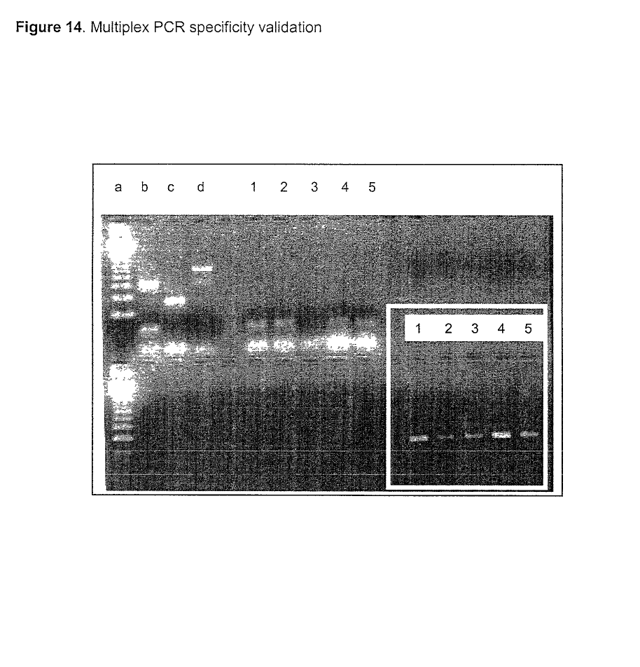

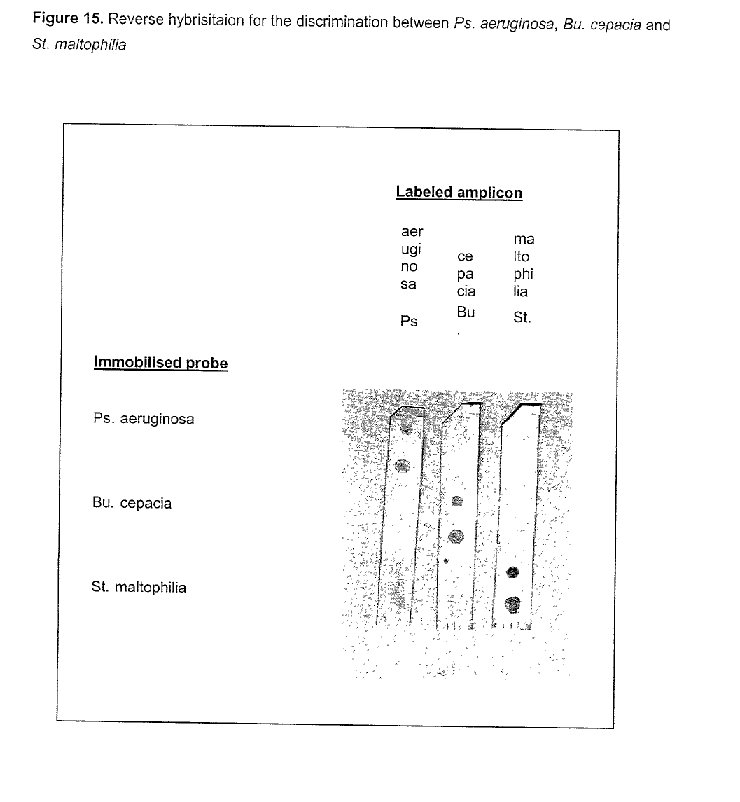

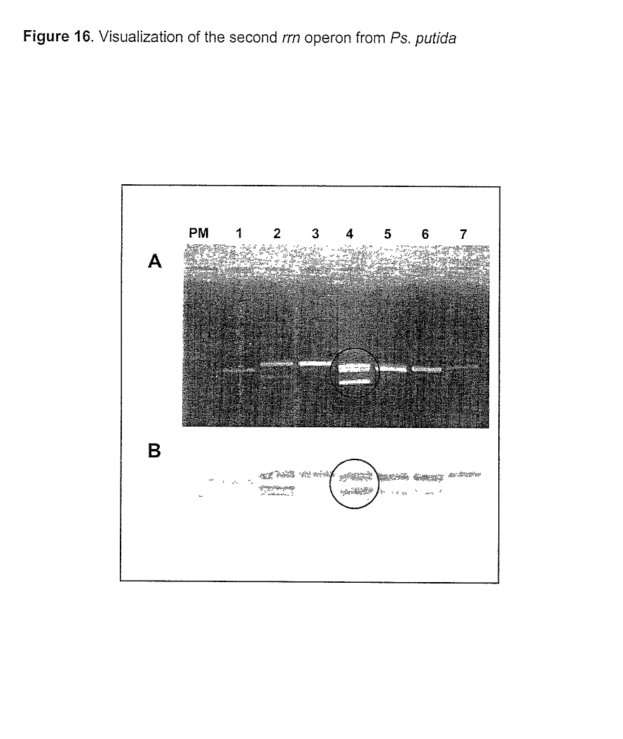

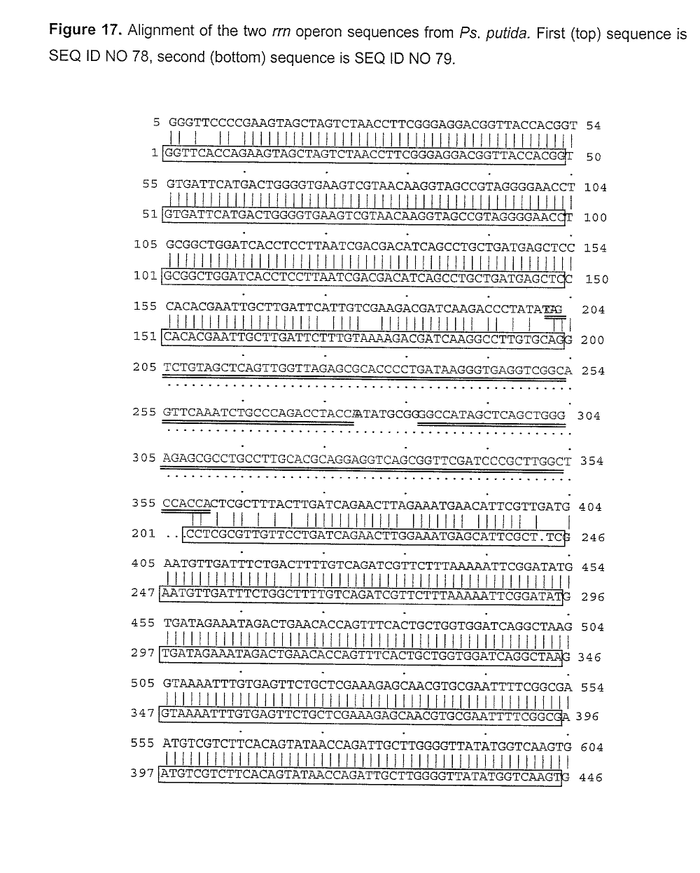

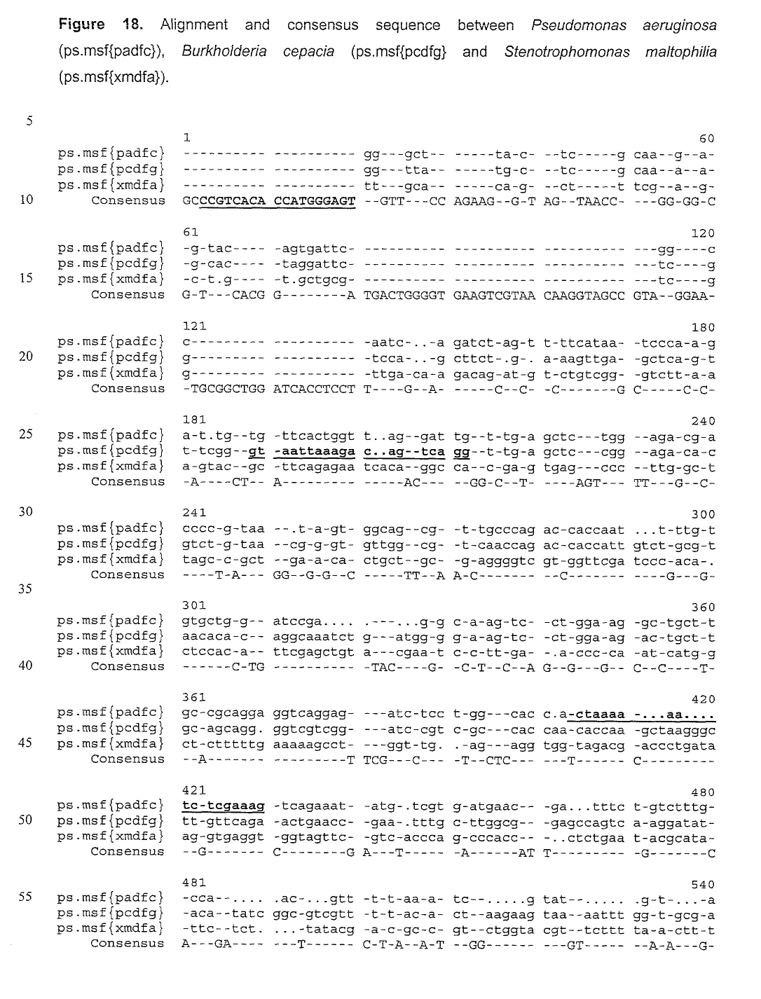

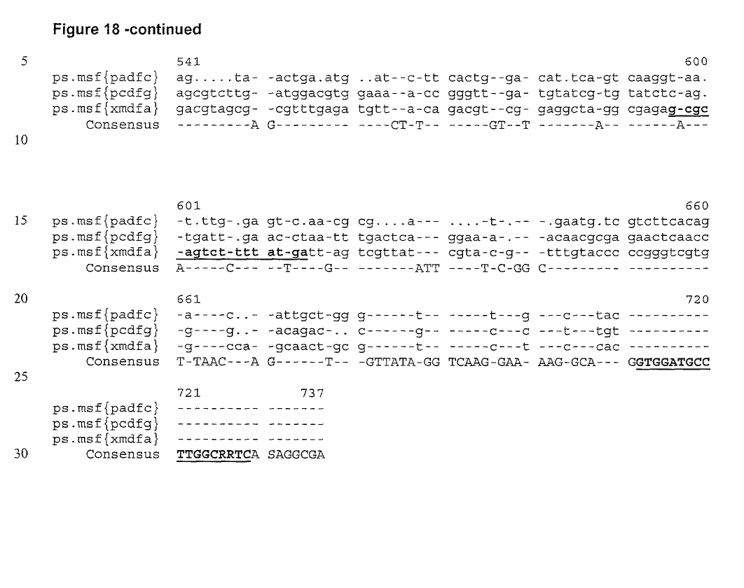

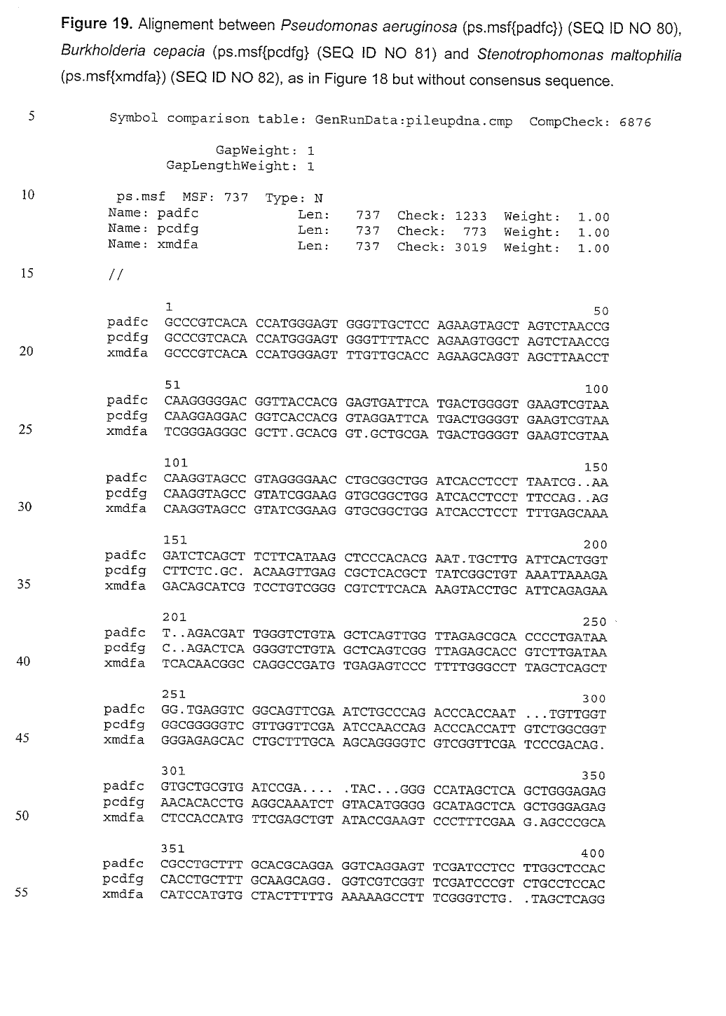

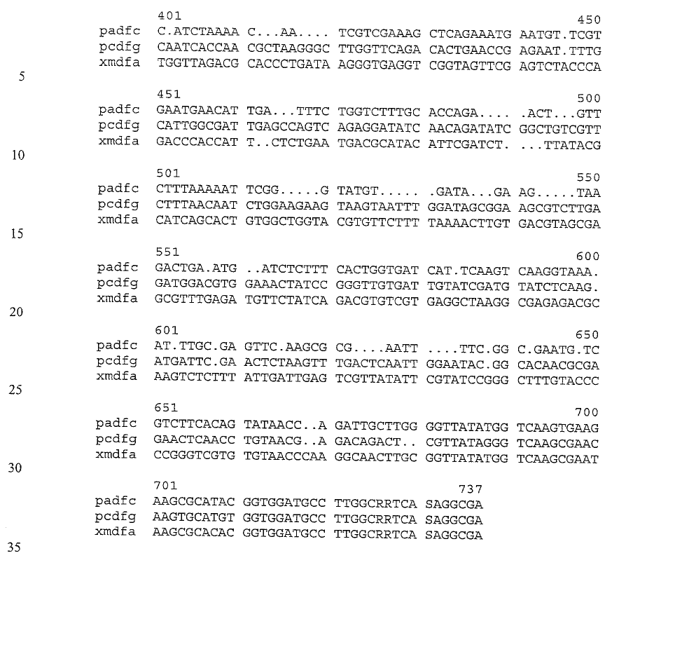

The first developed molecular strategy was an amplification by multiplex PCR allowing the production of fragments of different sizes according to the species from which DNA was amplified (FIG. 12). The sensibility and the specificity of this first strategy was validated on clinical strains (FIG. 13 and 14).

The second strategy comprised the development of a reverse hybridization system wherein the labeled and amplified DNA is specifically hybridized to probes corresponding to the different species (FIG. 15). The feasibility of this strategy allows to consider bioships/microarray application.

4. Organization of the rrn gene in Pseudomonas putida

In order to extend the amplification strategies by multiplex PCR and reverse hybridization to other species from the Ps. aeruginosa group, an amplification of the 3′end of 16S tRNA to the 5′ end of 23S tRNA was performed with DNA from control strains of Ps. putida, Ps. fluorescens, Ps. alcaligenes and Ps. pseudoalcaligenes, using PsSeql and PsSeq2 primers. Surprisingly the inventors noticed that for Ps. putida two bands of equal intensity but of different sizes were obtained (FIG. 16A). The presence of this second band of smaller size was confirmed for two other isolates of Ps. putida (clinical samples form different origin). A hybridization was performed using a probe produced by DNA amplification of Ps. aeruginosa (primers PsSeql and PsSeq2). The calorimetric detection of the hybrids confirmed that this smaller band was specific of the rrn gene (FIG. 16B). Two hypotheses were considered to explain the presence of this band:

the presence of a second hybridization site for one of the primers

the presence of two rrn operon of different sizes;

To check these two hypotheses, the two fragments were cloned and sequenced. The sequences confirmed that in Ps. putida, two rrn operons are present, one of which has a deletion. This deletion covers the region coding for the two tRNA corresponding to Alanine and Isoleucine amino acids (FIG. 17). Based on this knowledge, methods and primers for differential diagnosis are designed.

5.Alignment of the sequences of the three studied species

a) Primers used for the multiplex PCR:

The multiplex PCR amplifies distinctly Pseudomonas aeruginosa, Burkholderia cepacia and Stenotrophomonas maltophilia.

The primers represented in Table 6 have been designed by the present inventors. <<Psmulti(PsSeq1) and <<PsSeq2>> are universal primers for Pseudomonas (hybridizing effectively to a consensus region determined by the alignment of the three sequences). The three other primers <<Bcep >>, (<Paer >> and <<Smal >> are species-specific (c.f. FIG. 18 and FIG. 12A).

The amplification of the conserved region on the 16S rRNA gene was used as a control in most of the experiments. These sequences are represented in Table 7 and are known in the art.

b) Length of the amplified fragments (c.f. FIG. 12)

| | |
|---|---|
| PsSeq1-PsSeq2: | 726 bp |
| Bu. cepacia: | 209 bp |
| Ps. aeruginosa: | 427 bp |
| St. Maltophilia: | 612 bp |
| ProUn1-ProUn2: | 211 bp |

c) Code used for the alignments

ps.msf(padfc) =Pseudomonas aeruginosa

ps.msf(pcdfg) =Burkholderia cepacia

ps.msf(xmdfa) =Stenotrophoromonas maltophilia

d) Parameters used for the alignments in FIGS. 18 and 19

Plurality: 3.00 Threshold: 1 AveWeight: 1.00 AveMatch: 1.00 AvMisMatch: 0.00

### REFERENCES

1. Portaels f. Epidemiology of mycobacterial diseases. Clinics in Dermatology 1995 ;13: 207-222.

2. Raviglione M C, Snider D E, Kochi A. Global epidemiology of tuberculosis: morbidity and mortality of a wordlwide epidemic. JAMA 1995 ;273 :220-26.

3. Cobelens F G, van Deutekom H, Draayer-Jansen I W, et al. Risk of infetion with Myvccobacterium tuberculosis in travellers to areas of high tuberculosis endemicity=. Lancet 2000 ;356 :461-465.

4. Pablos-Mendez A, raviglione MC, Laszlo A, et al; Global surveillance for anti-tuberculosis-drug resistance, 1994-1997. New Engl J Med 1998;338 :1641-49.

5. Tsang A Y and Farber E R. The primary isolation of Mycobacterium ulcerans. Am. J. Clin. Pathol. 1973; 59: 688-692.

6. Portaels F, Fonteyne PA, De Beenhouwer H, de Rijk P, Guedenon A, Hayman J, Meyers WM. Variability in 3′ end of 16S rRNA sequence of Mycobacterium ulcerans is related to geographic origin of isolates. J. Clin Microbiology 1996 ;34 :962-965.

7. O'Callaghan E M, Tanner M S, Boulnois G J. Developpement of a PCR. probe test for identifying P.aeruginosa and P.(B) cepacia. Clin Pathol 1994; 47:222-226.

8. Karpati F and Jonasson J. Polymerase chain reaction for the detection of Pseudomonas aeruginosa, Stenotrophomonas maltophilia and Burkholderia cepacia in sputum of patients with cystic fibrosis. Molecular and Cellular probes, 1996, 10 :397-403.

9. Tyler S D, Strathdee C A, Rozee K R and Johnson WM. Oligonucleotide primers designed to differentiate pathogenic pseudomonads on the basis of the sequencing of genes coding for 16S-23S rRNA internal transcribed spacers. Clinical and Diagnostic Laboratory Immunology 1995 448453.

TABLE 1
Oligonucleotide primers
These consensus primers have been successfully
used to amplify a wide panel of reference and
clinical mycobacterial strains (see Table 4)

| | |
|---|---|
| U1: 5′-GAGTAGGTCATGGCTCCTCC-3′ (antisense) | (SEQ ID |
| | NO 1) |
| U6: 5′-GTGCGCATATAGCGGTCGTC-3′ (sense) | (SEQ ID |
| | NO 2) |
| U4: 5′-CATGCAGCGAATTAGAACGT-3′ (sense) | (SEQ ID |
| | NO 3) |

TABLE 2
Oligonucleotide Primers

| | |
|---|---|
| U2: 5′-AACTTGACGAACTCGCCG-3′ (sense) | (SEQ ID |
| | NO 4) |
| U3: 5′-AGGTATTCGCGCAGCATG-3′ (sense) | (SEQ ID |
| | NO 5) |
| U5: 5′-GTASGTCATRRSTYCTCC-3′ (antisense) | (SEQ ID |
| | NO 6) |
| U9: 5′-GGTGAACATTGGGCCGAA-3′ (antisense) | (SEQ ID |
| | NO 7) |

S = G or C; R = G or A; Y = T or C

U5 is an example of a degenerated primer that in combination with U4, as a consensus or universal primer, was used to amplify DNA fragments of various mycobacterial species. These mycobacterial species could subsequently be identified by reverse hybridization on a biochips

It is obvious that the above-mentioned nucleic acids can also be used as probes in other experiments.

Table 3. Oligonucleotide probes and primers

(a) Example of oligonucleotides used as capture probes that can specifically hybridize Mycobacteria strains on a biochips after DNA amplification using U4 and U5 consensus primers

For some species, several potential capture probes have been designed. Further testing may reveal advantages of some with respect to the others.

| | | |
|---|---|---|
| Avium 1: | 5′-CGGTCGTCTCCGAAGCCCGCG-3′ | (SEQ ID NO 8) |
| Gastrii 1: | 5′-GATCGGCAGCGGTGCCGGGG-3′ | (SEQ ID NO 9) |
| Gastril 2: | 5′-GTATCGCGGGCGGCAAGGT-3′ | (SEQ ID NO 10) |
| Gastrii 3: | 5′-TCTGCCGATCGGCAGCGGTGCCGG-3′ | (SEQ ID NO 11) |
| Gastril 4: | 5′-GCCGGGGCCGGTATTCGCGGGCGG-3′ | (SEQ ID NO 12) |
| Gordonae 1: | 5′-GACGGGCACTAGTTGTCAGAGG-3′ | (SEQ ID NO 13) |
| Intracellulare 1: | 5′-GGGCCGCCGGGGGCCTCGCCG-3′ | (SEQ ID NO 14) |
| Intracellulare 2: | 5′-GCCTCGCCGCCCAAGACAGTG-3′ | (SEQ ID NO 15) |
| Leprae 1: | 5′-GATTTCGGCGTCCATCGGTGGT-3′ | (SEQ ID NO 16) |
| Kansasi 1: | 5′-GATCGTCGGCAGTGGTGACGG-3′ | (SEQ ID NO 17) |
| Kansasi 2: | 5′-TCGTCGGCAGTGGTGAC-3′ | (SEQ ID NO 18) |
| Kansasi 3: | 5′-ATCCGCCGATCGTCGGCAGTGGTGACG-3′ | (SEQ ID NO 19) |
| Malmoense 1: | 5′-GACCCACAACACTGGTCGGCG-3′ | (SEQ ID NO 20) |
| Marinum 1: | 5′-CGGAGGTGATGGCGCTGGTCG-3′ | (SEQ ID NO 21) |
| Scrofulaceum 1: | 5′-CGGCGGCACGGATCGGCGTC-3′ | (SEQ ID NO 22) |
| Simiae 1: | 5′-ATCGCTCCTGGTCGCGCCTA-3′ | (SEQ ID NO 23) |
| Szulgai 1: | 5′-CCCGGCGCGACCAGCAGAACG-3′ | (SEQ ID NO 24) |
| Tuberculosis 1: | 5′-GCCGTCCAGTCGTTAATGTCGC-3′ | (SEQ ID NO 25) |
| Xenopi 1: | 5′-CGGTAGAAGCTGCGATGACACG-3′ | (SEQ ID NO 26) |

It is obvious that the above-mentioned nucleic acids can also be used as primers in other experiments.

(b) Example of oligonucleotides used as capture probes that can specifically hybridize Mycobacteria strains on a biochips after DNA amplification using Ul and U4 consensus primers

For some species, several potential capture probes have been designed. Further testing may reveal advantages of some with respect to the others.

| | | |
|---|---|---|
| Avium 2: | 5′-GCGCGGTCGTCTCCGAAGCCC-3′ | (SEQ ID NO 27) |
| Avium 3: | 5′-CCGCTCGGCACTAAAAGGCAGTGGAAGC-3′ | (SEQ ID NO 28) |
| Avium 4: | 5′-GAAGCCCGCGGGCAAGCCAAT-3′ | (SEQ ID NO 29) |
| Gastrii 1: | 5′-GATCGGCAGCGGTGCCGG-3′ | (SEQ ID NO 30) |
| Gastrii 5: | 5′-GCGGTGCCGGGGCCGGTA-3′ | (SEQ ID NO 31) |
| Gastrii 6: | 5′-CGGTATCGCGGGCGGCAAGGT-3′ | (SEQ ID NO 32) |
| Gordonae 2: | 5′-GGCGACGGGCACTAGTTGTCAGAGGTG-3′ | (SEQ ID NO 33) |
| Intracellulare 3: | 5′-CCGCCGGGGGCCTCGC-3′ | (SEQ ID NO 34) |
| Intracellulare 4: | 5′-TCGCCGCCCAAGACAGTGGCG-3′ | (SEQ ID NO 35) |
| Kansasii 4: | 5′-ATCCGCCGATCGTCGGCAGTGGT-3′ | (SEQ ID NO 36) |
| Kansasii 5: | 5′-GATCGTCGGCAGTGGTGACGGGG-3′ | (SEQ ID NO 37) |
| Kansasii 6: | 5′-GGGCCGGTATCACGGGGGCAA-3′ | (SEQ ID NO 38) |
| Leprae 2: | 5′-GATTTCGGCGTCCATCGGTGGTAG-3′ | (SEQ ID NO 39) |
| Malmoense 2: | 5′-AACGCAAGATCTCGAAGGTGTTTTCAAAGGCG-3′ | (SEQ ID NO 40) |
| Malmoense 3: | 5′-GACCCACAACACTGGTCGGCGCC-3′ | (SEQ ID NO 41) |
| Marinum 2: | 5′-GCCAATCGGCTCGGCGGGA-3′ | (SEQ ID NO 42) |
| Marinum 3: | 5′-ATCGACGGAGGTGATGGCGCTG-3′ | (SEQ ID NO 43) |
| Simiae 2: | 5′-CGATCGCTCCTGGTCGCGCCT-3′ | (SEQ ID NO 44) |
| Simiae 3: | 5′-CCGGCGCACCCGCTCGAAC-3′ | (SEQ ID NO 45) |
| Szulgai 2: | 5′-CTGCGATGAGCAAGCGGCCCG-3′ | (SEQ ID NO 46) |
| Szulgai 3: | 5′-GCGGCCCGGTCGGCCG-3′ | (SEQ ID NO 47) |
| Tuberculosis 2: | 5′-CGGCCGTCCAGTCGTTAATGTCGC-3′ | (SEQ ID NO 48) |
| Xenopi 2: | 5′-CGGTAGAAGCTGCGATGACACGCCA-3′ | (SEQ ID NO 49) |

It is obvious that the above-mentioned nucleic acids can also be used as primers in other experiments.

TABLE 3
(c) Example of oligonucleotides used as species specific primers
The specific primer pair includes an universal (consensus) antisense
primer (for instance U1, U5 or U9) and one of the following primers.
This can be used for amplifying mycobacterial DNA in a multiplex
diagnostic strategy. Theoretical annealing temperature (Tm) of each
primer as well as the expected size of the amplicon are given.

| | | | |
|---|---|---|---|
| U1 | 5′-GAGTAGGTCATGGCTCCTCC-3′ | | Tm 63,7 (see |
| | | | table 1) |
| Avium 5: | 5′-GCAAGCCAATGGCGA-3′ | (SEQ ID NO 50) | Tm 64,8 |
| | 166 bp | | |
| Intracellulare 5: | 5′-CTCGCCGCCCAAGA-3′ | (SEQ ID NO 51) | Tm 65,1 |
| | 172 bp | | |
| Tuberculosis 3: | 5′-CCGTCCAGTCGTTAATGTC-3′ | (SEQ ID NO 52) | Tm 61,6 |
| | 134 bp | | |
| Simiae 4: | 5′-ACGATCGCTCCTGGTCG-3′ | (SEQ ID NO 53) | Tm 66,6 |
| | 219 bp | | |
| Malmoense 4: | 5′-AAGATCTCGAAGGTGTTTTCAA-3′ | (SEQ ID NO 54) | Tm 62,5 |
| | 119 bp | | |

Mycobacterium avium can be further distinguished from Mycobacterium avium subspecies paratuberculosis by co-amplification with F57 gene specific primers. A specific primer pair for amplification of at least part of the F57 gene of Mycobacterium avium subspecies paratuberculosis is:

| | |
|---|---|
| F57a | |
| 5′-GGTCGCGTCATTCAGAATC-3′ (SEQ ID NO 55) | Tm 64,4 |
| 439 bp | |
| F57b | |
| 5′-TCTCAGACAGTGGCAGGTG-3′ (SEQ ID NO 56) | Tm 64,1 |

Other F57 primers can be used such as MPT1 (internal primer), which in combination with F57b generates a fragment of 618 bp with a sequence represented in SEQ ID NO 75.

It is obvious that the above-mentioned nucleic acids can also be used as probes in other experiments.

TABLE 4
Validation of the consensus U1-U4 amplification on clinical
mycobacterial strains.

| | | Nbr of + amplif./ |
|---|---|---|
| Mycobacteria | size (bp) | Nbr of samples |
| Tuberculosiss Group (TUB) | | |
| M. tuberculosis | 177 | 15/15 |
| M. bovis | 177 | 9/10 |
| M. africanum | 177 | 5/5 |
| Non Tuberculosis Group (NTB) | | |
| M. avium Complex (MAC) | | |
| M. avium | 256 | 16/17 |
| M. paratuberculosis | 256 | 21/21 |
| M. intracellulare | 256 | 9/9 |
| M. scrofulaceum | 259 | 4/5 |
| MOTT (Myc. Other Then Tub-MOTT) | | |
| M. szulgai | 163 | 9/9 |
| M. kansasii | 225 | 10/11 |
| M. gastri | 223 | 2/2 |
| M. xenopi | 265 | 12/12 |
| M. ulcerans | 211 | 20/20 |
| M. leprae | 269 | 1/1 |
| M. non chromogenicum | 169 | 2/2 |
| M. simiae | 298 | 6/11 |
| M. malmoense | 290 | 4/7 |
| M. gordonae | 182 | 4/10 |
| M. marinum | 236 | 5/5 |

TABLE 5
Classification of Pseudomonas

| | |
|---|---|
| Group I | Pseudomonas fluorescens (P. aeruginosa; P. fluorescens; |
| | P. putida) |
| | Pseudomonas stutzeri |
| | Pseudomonas pseudoalcaligenes; Ps. alcaligenes |
| Group II | Burkholderia cepacia; B. mallei; B. pseudomallei; B. pickettii; |
| | B. gladioli |
| Group III | Comanomas testosteroni; C. acidovrans |
| Group IV | Brevendimonas diminuta; B. vesicularis |
| Group V | Stenotrophomonas maltophilia |

TABLE 6
Species specific and universal probes

| Name | Sequence | | | T°m |
|---|---|---|---|---|
| PsMulti | 5′ ACGTCACACCATGGGAGT 3′ | (SEQ ID NO 83) | Sense | 62.8° C. |
| (PsSeq1) | | | | |
| Bcep | 5′ CCCTGAGTCTGTCTTTAATTTAC 3′ | (SEQ ID NO 84) | Antisense | 58.3° C. |
| Paer | 5′ CTTTCGACGATTGTTTTAGT 3′ | (SEQ ID NO 85) | Antisense | 56.8° C. |
| Smal | 5′ TCAATAAAAGAGACTTGCGTC 3′ | (SEQ ID NO 86) | Antisense | 57.9° C. |
| PsSeq2 | 5′ GATTGCCAAGGCATCCAC 3′ | (SEQ ID NO 87) | Antisense | 65.4° C. |

TABLE 7
Control probes

| | | | |
|---|---|---|---|
| ProUni1 | 5′ GAGGAAGGTGGGGATGAC 3′ (SEQ ID NO 88) | Sense | 63.1° C. |
| ProUni2 | 5′ TGGGAACGTATTCACCGT 3′ (SEQ ID NO 89) | Antisense | 62.5° C. |

1. Method for detecting at least one Mycobacterium strain in a sample, comprising:
(i) providing at least one Mycobacterium species-specific upstream p34 gene region (us-p34) nucleotide probe,
(ii) reacting said us-p34 nucleotide probe with said sample under conditions that allow for the selective formation of nucleotide duplexes between said us-p34 nucleotide probe and a corresponding Mycobacterium nucleic acid target present in said sample, and,
(iii) detecting any nucleotide duplexes containing said us-p34 nucleotide probe.

2. Method according to claim 1 wherein said Mycobacterium species-specific us-p34 nucleotide probe specifically hybridizes with at least part of a sequence selected from SEQ ID NOs 57 to 74, or the complement thereof, or the corresponding sequences wherein T has been replaced by U.

3. Method according to claim 1 wherein said Mycobacterium species-specific us-p34 nucleotide probe is selected from the group of sequences represented in SEQ ID NOs 8 to 54 and SEQ ID NOs 57 to 74, or the complement thereof, or the corresponding sequences wherein T has been replaced by U.

4. Method for the differential detection of Mycobacteria in a sample, comprising:
(i) providing at least two distinct Mycobacterium species-specific us-p34 nucleotide probes,
(ii) reacting said us-p34 nucleotide probes with said sample under conditions that allow for the selective formation of nucleotide duplexes between said us-p34 specific nucleotide probe and a Mycobacterium nucleic acid present in said sample,
(iii) detecting any nucleotide duplexes containing said us-p34 nucleotide probe, and,
(iv) inferring from the nucleotide duplex formed, the presence and the identification of a specific Mycobacterium strain.

5. Method according to claim 4 wherein said Mycobacterium species-specific us-p34 nucleotide probes are selected from the group of sequences represented in SEQ ID NOs 8 to 54 and SEQ ID NOs 57 to 74.

6. Method for detecting at least one Mycobacterium strain in a sample, comprising:
(i) providing at least one suitable primer pair comprising a sense or antisense Mycobacterium species-specific us-p34 primer,
(ii) reacting said us-p34 primer pair with said sample under conditions that allow for the selective amplification of an us-p34 sequence in a Mycobacterium nucleic acid present in said sample, and,
(iii) detecting the amplified product of step (ii), and,
(iv) inferring from the amplification product the presence and the identification of at least one specific Mycobacterium strain.

7. Method according to claim 6 wherein the sense or antisense Mycobacterium species-specific us-p34 primer is selected from the group of sequences represented in SEQ ID NOs 8 to 54.

8. Method for the differential detection of mycobacteria in a sample, comprising:
(i) providing at least one suitable us-p34 primer pair containing a sense or anti-sense us-p34 primer,
(ii) reacting said us-p34 primer pair with said sample under conditions that allow for the selective amplification of us-p34 sequences of at least one Mycobacterium nucleic acid present in said sample,
(iii) detecting the amplified product of step (ii), and,
(iv) inferring from the amplified product formed, the presence and the identification of at least one (specific) Mycobacterium.

9. Method according to claim 8 wherein said us-p34 primer pair is selected from the group of sequences represented in SEQ ID NOs 1 to 54.

10. Method for the detection of MAC complex Mycobacterium species in a sample, comprising:
(i) providing at least one us-p34 probe selected from the group of sequences represented in SEQ ID NOs 8, 14, 15, 22, 27, 28, 29, 34, 35, 50, 51, 57, 68, and 73,
(ii) reacting said us-p34 probe with said sample under conditions that allow for the selective formation of nucleotide duplexes between said us-p34 nucleotide probe and a MAC complex Mycobacterium nucleic acid target in said sample, and,
(iii) detecting any nucleotide duplexes containing said us-p34 nucleotide probe.

11. Method for the detection of MOTT Mycobacterium species in a sample, comprising:
(i) providing at least one us-p34 probe selected from the group of sequences represented in SEQ ID NOs 9 to 13, 16 to 21, 24, 25, 26, 30 to 33, 36 to 47, 49, 53, 54, 59 to 64, 67, 69 to 72 and 74,
(ii) reacting said us-p34 primer probe said sample under conditions that allow for the selective formation of nucleotide duplexes between said us-p34 nucleotide probe and a MOTT Mycobacterium nucleic acid target in said sample, and,
(iii) detecting any nucleotide duplexes containing said us-p34 nucleotide probe.

12. Method for detecting new us-p34 sequences in a sample, comprising:
(i) providing at least one suitable primer pair comprising a sense and anti-sense us-p34 primer selected from the sequences represented in SEQ ID NOs 1 to 7,
(ii) reacting said us-p34 primer pair with said sample under conditions that allow for the amplification of an us-p34 sequence in a Mycobacterium nucleic acid target in said sample, and,
(iii) determining the sequence of the amplification product obtained in (ii).

13. Method for the differential detection of mycobacteria in a sample, comprising:
(i) providing at least one suitable primer pair comprising a sense and anti-sense us-p34 primer selected from SEQ ID NOs 1 to 7,
(ii) reacting said us-p34 primer pair with said sample under conditions that allow for the amplification of an us-p34 sequence in a Mycobacterium nucleic acid target in said sample,
(iii) selectively hybridizing the amplification products obtained in (ii) with at least one Mycobacterium species-specific us-p34 nucleotide probe selected from the group of sequences represented in SEQ ID NOs 8 to 74,
(iv) detecting any nucleotide duplexes containing said Mycobacterium species-specific us-p34 nucleotide probe, and,
(v) inferring from the nucleotide duplex formed, the presence of a specific Mycobacterium species.

14. A Mycobacterium species-specific us-p34 nucleotide probe or primer comprising at least 8 contiguous nucleotides from one of the nucleic acid sequences represented in SEQ ID NOs 57 to 74, or the complement thereof, or the corresponding sequences wherein T has been replaced by U.

15. The Mycobacterium species-specific us-p34 nucleotide probe or primer of claim 14 selected from the sequences as represented in SEQ ID NOs 8 to 54.

16. A Mycobacterium us-p34 nucleotide primer selected from the sequences as represented in SEQ ID NOs 1 to 7.

17. A nucleic acid comprising a sequence selected from SEQ ID NOs 8 to 54, 57 to 64, 66, 67 and 69 to 74.

18. A composition comprising at least one nucleotide probe, primer or sequence according to any of claims 14 to 17.

19. A diagnostic kit comprising a probe, primer or sequence according to any of claims 14 to 17 or a composition according to claim 18.

20. A solid support for the detection of mycobacteria comprising fixed to said support at least two capture probes selected from SEQ ID NOs 1 to 54 and 57 to 74.

21. A solid support according to claim 19 for use in a method of any of claims 1 to 5 or 13.

22. A method for differentiating between Mycobacterium bovis and Mycobacterium tubercolusosis in a sample, comprising:
(i) providing at least one us-p34 probe selective for Mycobacterium bovis or Mycobacterium tuberculosis wherein said probe is SEQ ID NO 66 for Mycobacterium bovis or SEQ ID NO 65 for Mycobacterium tuberculosis,
(ii) reacting said us-p34 nucleotide probe with said sample under conditions that allow for the selective formation of nucleotide duplexes between said us-p34 nucleotide probe and a corresponding Mycobacterium bovis or tuberculosis nucleic acid target present in said sample, and,
(iii) detecting any nucleotide duplexes containing said Mycobacterium bovis or tuberculosis specifc us-p34 nucleotide probe.

23. A method for differentiating between Mycobacterium bovis and Mycobacterium tubercolusosis in a sample, comprising:
(i) providing at least one suitable primer pair comprising at least one sense or antisense us-p34 primer selective for Mycobacterium bovis or Mycobacterium tuberculosis wherein said primer is SEQ ID NO 66 for Mycobacterium bovis or SEQ ID NO 65 for Mycobacterium tuberculosis,
(ii) reacting said us-p34 primer pair with said sample under conditions that allow for the selective amplification of Mycobacterium bovis and/or Mycobacterium tuberculosis nucleic acid target present in said sample, and,
(iii) inferring from the reaction product(s) the presence of Mycobacterium bovis and/or Mycobacterium tuberculosis in said sample.

24. A method for differentiating between Mycobacterium avium and Mycobacterium avium subspecies paratuberculosis in a sample, comprising:
(i) providing at least one us-p34 probe selective for Mycobacterium avium and Mycobacterium avium subspecies paratuberculosis wherein said probe is selected from the sequences represented in SEQ ID NOs 8, 27 to 29, 50 or 58 for Mycobacterium avium or SEQ ID NO 68 for Mycobacterium avium subspecies paratuberculosis
(ii) reacting said us-p34 nucleotide probe with said sample under conditions that allow for the selective formation of nucleotide duplexes between said us-p34 nucleotide probe and a corresponding Mycobacterium avium and Mycobacterium avium subspecies paratuberculosis nucleic acid target present in said sample, and,
(iii) detecting any nucleotide duplexes containing said Mycobacterium avium and Mycobacterium avium subspecies paratuberculosis specifc us-p34 nucleotide probe

25. A method for differentiating between Mycobacterium avium and Mycobacterium avium subspecies paratuberculosis in a sample, comprising:
(i) providing at least one suitable primer pair comprising at least one sense or antisense us-p34 primer selective for Mycobacterium avium and Mycobacterium avium subspecies paratuberculosis wherein said primer is selected from the sequences represented in SEQ ID NOs 8, 27 to 29, 50 or 58 for Mycobacterium avium or SEQ ID NO 68 for Mycobacterium avium subspecies paratuberculosis,
(ii) reacting said us-p34 primer pair with said sample under conditions that allow for the selective amplification of Mycobacterium avium and Mycobacterium avium subspecies paratuberculosis nucleic acid target present in said sample, and,
(iii) inferring from the reaction product(s) the presence of Mycobacterium avium and Mycobacterium avium subspecies paratuberculosis in said sample.


SEQUENCE LISTING (89 sequences)

SEQ ID NO: 1 - DNA, 20 bp, Mycobacterium sp.
gagtaggtcatggctcctcc

SEQ ID NO: 2 - DNA, 20 bp, Mycobacterium sp.
catgcagcgaattagaacgt

SEQ ID NO: 3 - DNA, 20 bp, Mycobacterium sp.
catgcagcgaattagaacgt

SEQ ID NO: 4 - DNA, 18 bp, Mycobacterium sp.
aacttgacgaactcgccg

SEQ ID NO: 5 - DNA, 18 bp, Mycobacterium sp.
aggtattcgcgcagcatg

SEQ ID NO: 6 - DNA, 18 bp, Mycobacterium sp.
gtasgtcatrrstyctcc

SEQ ID NO: 7 - DNA, 18 bp, Mycobacterium sp.
ggtgaacattgggccgaa

SEQ ID NO: 8 - DNA, 21 bp, Mycobacterium avium
cggtcgtctccgaagcccgcg

SEQ ID NO: 9 - DNA, 20 bp, Mycobacterium gastri
gatcggcagcggtgccgggg

SEQ ID NO: 10 - DNA, 19 bp, Mycobacterium gastri
gtatcgcgggcggcaaggt

SEQ ID NO: 11 - DNA, 24 bp, Mycobacterium gastri
tctgccgatcggcagcggtgccgg

SEQ ID NO: 12 - DNA, 24 bp, Mycobacterium gastri
gccggggccggtattcgcgggcgg

SEQ ID NO: 13 - DNA, 22 bp, Mycobacterium gordonae
gacgggcactagttgtcagagg

SEQ ID NO: 14 - DNA, 21 bp, Mycobacterium intracellulare
gggccgccgggggcctcgccg

SEQ ID NO: 15 - DNA, 21 bp, Mycobacterium intracellulare
gcctcgccgcccaagacagtg

SEQ ID NO: 16 - DNA, 22 bp, Mycobacterium leprae
gatttcggcgtccatcggtggt

SEQ ID NO: 17 - DNA, 21 bp, Mycobacterium kansasii
gatcgtcggcagtggtgacgg

SEQ ID NO: 18 - DNA, 17 bp, Mycobacterium kansasii
tcgtcggcagtggtgac

SEQ ID NO: 19 - DNA, 27 bp, Mycobacterium kansasii
atccgccgatcgtcggcagtggtgacg

SEQ ID NO: 20 - DNA, 21 bp, Mycobacterium malmoense
gacccacaacactggtcggcg

SEQ ID NO: 21 - DNA, 21 bp, Mycobacterium marinum
cggaggtgatggcgctggtcg

SEQ ID NO: 22 - DNA, 20 bp, Mycobacterium scrofulaceum
cggcggcacggatcggcgtc

SEQ ID NO: 23 - DNA, 20 bp, Mycobacterium simiae
atcgctcctggtcgcgccta

SEQ ID NO: 24 - DNA, 21 bp, Mycobacterium szulgai
cccggcgcgaccagcagaacg

SEQ ID NO: 25 - DNA, 22 bp, Mycobacterium tuberculosis
gccgtccagtcgttaatgtcgc

SEQ ID NO: 26 - DNA, 22 bp, Mycobacterium xenopi
cggtagaagctgcgatgacacg

SEQ ID NO: 27 - DNA, 21 bp, Mycobacterium avium
gcgcggtcgtctccgaagccc

SEQ ID NO: 28 - DNA, 28 bp, Mycobacterium avium
ccgctcggcactaaaaggcagtggaagc

SEQ ID NO: 29 - DNA, 21 bp, Mycobacterium avium
gaagcccgcgggcaagccaat

SEQ ID NO: 30 - DNA, 18 bp, Mycobacterium gastri
gatcggcagcggtgccgg

SEQ ID NO: 31 - DNA, 18 bp, Mycobacterium gastri
gcggtgccggggccggta

SEQ ID NO: 32 - DNA, 21 bp, Mycobacterium gastri
cggtatcgcgggcggcaaggt

SEQ ID NO: 33 - DNA, 27 bp, Mycobacterium gordonae
ggcgacgggcactagttgtcagaggtg

SEQ ID NO: 34 - DNA, 16 bp, Mycobacterium intracellulare
ccgccgggggcctcgc

SEQ ID NO: 35 - DNA, 21 bp, Mycobacterium intracellulare
tcgccgcccaagacagtggcg

SEQ ID NO: 36 - DNA, 23 bp, Mycobacterium kansasii
atccgccgatcgtcggcagtggt

SEQ ID NO: 37 - DNA, 23 bp, Mycobacterium kansasii
gatcgtcggcagtggtgacgggg

SEQ ID NO: 38 - DNA, 21 bp, Mycobacterium kansasii
gggccggtatcacgggggcaa

SEQ ID NO: 39 - DNA, 24 bp, Mycobacterium leprae
gatttcggcgtccatcggtggtag

SEQ ID NO: 40 - DNA, 32 bp, Mycobacterium malmoense
aacgcaagatctcgaaggtgttttcaaaggcg

SEQ ID NO: 41 - DNA, 23 bp, Mycobacterium malmoense
gacccacaacactggtcggcgcc

SEQ ID NO: 42 - DNA, 19 bp, Mycobacterium marinum
gccaatcggctcggcggga

SEQ ID NO: 43 - DNA, 22 bp, Mycobacterium marinum
atcgacggaggtgatggcgctg

SEQ ID NO: 44 - DNA, 21 bp, Mycobacterium simiae
cgatcgctcctggtcgcgcct

SEQ ID NO: 45 - DNA, 19 bp, Mycobacterium simiae
ccggcgcacccgctcgaac

SEQ ID NO: 46 - DNA, 21 bp, Mycobacterium szulgai
ctgcgatgagcaagcggcccg

SEQ ID NO: 47 - DNA, 16 bp, Mycobacterium szulgai
gcggcccggtcggccg

SEQ ID NO: 48 - DNA, 24 bp, Mycobacterium tuberculosis
cggccgtccagtcgttaatgtcgc

SEQ ID NO: 49 - DNA, 25 bp, Mycobacterium xenopi
cggtagaagctgcgatgacacgcca

SEQ ID NO: 50 - DNA, 15 bp, Mycobacterium avium
gcaagccaatggcga

SEQ ID NO: 51 - DNA, 14 bp, Mycobacterium intracellulare
ctcgccgcccaaga

SEQ ID NO: 52 - DNA, 19 bp, Mycobacterium tuberculosis
ccgtccagtcgttaatgtc

SEQ ID NO: 53 - DNA, 17 bp, Mycobacterium simiae
acgatcgctcctggtcg

SEQ ID NO: 54 - DNA, 22 bp, Mycobacterium malmoense
aagatctcgaaggtgttttcaa

SEQ ID NO: 55 - DNA, 19 bp, Mycobacterium avium subspecies paratuberculosis
ggtcgcgtcattcagaatc

SEQ ID NO: 56 - DNA, 19 bp, Mycobacterium avium subspecies paratuberculosis
tctcagacagtggcaggtg

SEQ ID NO: 57 - DNA, 216 bp, Mycobacterium intracellulare
gttctacctgtgctgagcaagctccggtgataccgaccgtctcgccggagggccgccgggggcctcgccgcccaagacagtggcggcgccaccggttcccgcacgtgcgctagcgtgggtgatcgaccgcgtcgcaatgcggtgacgcgcctgcaagcacagcgtcgcatcgccaccgcggcgcccgctcggcacttaaaggcactggtagcaaca

SEQ ID NO: 58 - DNA, 881 bp, Mycobacterium avium
tcgtagctggcttcctcgtcggtccacagcgcccgcatcgcttccaggtattcgcgcagcatggtgcggcgccggcccgccggcacgccgtggtcggcgagttcgtcggtgttccagccgaacccgacgccgaggctgacccggccgccggacagatggtcaagggtggcaatacttttcgccagcgtgatcgggtcgtgttcgaccggcagggccaccgcggtggacagccgcacccgcgaggtgacggcacaggccgcgcccagactgacccacgggtccagggtgcgcatgtagcggtcgtcgggcagcgacgcgtcgccggtggtcgggtgcgcggcctcccgcttgatcgggatatgcgtgtgttccggcacgtagaaggtcgcaaacccgtggtcgtcggcaagcttcgcggccgcagccggagagatgccacggtcgctggtgaaaagcacaagcccgtaatccatgcagtgaattagaacgtgttctacctctgcggggcaagctgtcgtgatacggaccgtctcgccgcgcggtcgtctccgaagcccgcgggcaagccaatggcgacggcaccggccgtcgcacgtgcgctagcgtgggtgatcgaccgtgtcgctcgcgcagtgacgcgcctgcaagcaccgcgtcgcatcgcaaccgtggcgcccgctcggcactaaaaggcagtggaagcaacaggaggagccatgacctactctcccggcagccccggatatccaccggcgcagtctggcggcacctatgcaggcgccacaccatctttcgccaaagacgacgacggcaagagcaaactcccgctctacctcaacatcgccgtggtcgccctgggtttcgcggcctacctgctgaat

SEQ ID NO: 59 - DNA, 642 bp, Mycobacterium gastri
gtgcgccggcgccccggcggcacgccatggtcggcgagttcgtgcgcccggcggcacgccatggtcggcgagttcgtcggtgttccagccgaatccgacgccgacgctgacccggcccccggatagtggtccagcgtggcaatgcttttggccagcgtgatcgggtcatgctccaccgcagcgcaaccgcggttgacagcctgactcgggaggtgaccgctgaagccgcacccaagctcacccacgggtccagggtgcgcatatagcggtcgtccggcagcgacgcgtcacccgtcgtgggatgggcggcttcccgtttgaccgggatatgcgtgtgttcgggcacgtagagagtgcgaaagccatggtcgtcggccagtttcgcggctgccgccggggagatcccacggtcgctggtgaaaaggacaagcccgtaatccatgaacagaattagaacgtgttctacctccgccgggcaagcggctcatctgccgatcggcagcggtgccggggccggtatcgcgggcggcaaggtcgccacggcgtgagtacccggccgtgcgctagcgtgggtcatcgaattgtgtcgcagggagcaatcgtcgcattgcagcaggcgtagcgacggcaccggaggtaaca

SEQ ID NO: 60 - DNA, 745 bp, Mycobacterium gordonae
gtgcgacgacggccggccagcacgttatggtcggcgagctcgtcggtgttccagccgaacccgacgccgaggctaactcgcccgccggacaggtgatccagcgtggcgatgcttttcgccaaggtgatcgggtcatgctcgaccggcaacgcgactgccgtcgacagccgcacccgcgacgtcacagcacacgccgcgcccaggctcacccagggatccagggtgcgcatataacggtcgtcgggcagcgtctcgtctccggtggtgggatgagccgcctcgcgtttgatcgggatatgcgtgtgttcgggtacgtagaaggtgtgaaaaccatgtgtgtcggcaagtttcgctgctgccgcaggggaaataccgcgatcgctggtgaacagaacgaggctgtagtccatgccccaatttagaacgtgttctacttttggccgcagccgaccccctgcggcgacgggcactagttgtcagaggtgcgctagcgtggttgatcgaatgcgtcgcaggccgtaccgcgtcgtgccgaagcagaggggccgtgacggcaccggaagcaacaggaggacttatgacctacccgcccggtagtcccggatatccatccgcccagcagtcggccggcaactacggcagctccgctcccgccgccggccagtccgagccgggtgaaagcaagctgggactgtacctggccatcgcggtggcggccctgggcctactggcgtacctcttcagc

SEQ ID NO: 61 - DNA, 785 bp, Mycobacterium kansasii
gtgcgccggcgcgccggcggcacgccatggtcagcgagttcgtcggtgttccagccgaatccgacgccgacgctgacccgccccccggataggtggtccagcgtggcaatgcttttggccagcgtgatcgggtcatgctcgaccggcaacgcaaccgctgttgacagtcggacccggaaggtgaccgctgaagccgcgcccaaactcacccacgggtccagcgtgcgcatatagcggtcgtccggcagcgacgcgtcacccgtcgtgggatggcggcctcccgtttgaccgggatgtgcgtgtgttcgggcacgtagaaagtgcgaaagccatggtcgtcggccagtttcgcggctgccgcgggagaaatgccacggtcgctggtgaaaaggacaagcccgtaatccatgaacagaattagaacgtgttctacctcagccgggcaagcggctcatccgccgatcgtcggcagtggtgacggggccggtatcacgggggcaaggtcgccacggcgcgagtaccaggccgtgcgctagcgtgggtcatcgaatcgtgtcgcagggagcaatcgtcgcattgcagcaggcgtagcgacggcactggaggtaacaggaggagccatgacctactcaccaggtagtcccggatatccgcccgcgcaatcggccggctcctacggagccgccacaccgtctttcgccaaggccgacgacggtgtcagcaagcttccgatgtacctgagcatggcggttgccgcgctcgggctgctggcgtatctggccagc

SEQ ID NO: 62 - DNA, 691 bp, Mycobacterium malmoense
tcgtaggccgcttcctcctgggtccacagcgcccgcattgcctcgatgtattcacgcagcatggtgcgacggcgcccggccggcacgccgtggtcggcgagctcgtcggtgttccagccaaacccaacgccgaggctgacccggccgccggacaggtggtccaaggtggcaatacttttcgccagcgtgatcgggtcgtgctcgacgggcagcgccaccgcggtagacagccgcacccgcgacgtcacggcgcacgccgcgcccaggctcacccacgggtctagcgtgcgcatatagcggtcgtccggcaagcgacgcgccacccgtcgtcggatgggccgcctcgcgcttgaccgggatatgggtgtgttccggcacgtagaacgtctggaagccgtggtcgtcggcaagtttggcggctgccgccggggagatgccgcggtcgctggtgaaaagtacaagcccgtaatccatggacagaattagaacgtgttctaccggcggtgggcaagccgctgcgccgccgaggatctcgactcggacccacaacactggtcggcgccgggcgcgccgacaggtcggtcggcccggcacgggcggccgaacgtgcgctagcgtgggtgatcgatcgcgtcgcaacgcaagatctcatgcggcgtcgctgagggtcttgaaggcactggaagcaata

SEQ ID NO: 63 - DNA, 698 bp, Mycobacterium simiae
tcgtattgggcttcttcctgcgtccacagcgcccgcatggcttccaggtactcgcgcagcatggtccgccggcgcgccggcggcacgttgtggtcggccagttcgtcggtgttccaaccgaacccgacgcccacactgacccgtccgccggacagatggtccagggtggcgatgcttttcgccagcgtgatcgggtcgtgctcgacgggcagcgcgaccgcggtggacagtcgcacccgcgaggtgaccgcgcacgccgcgcccagactgacccacgggtccagcgtgcgcatgtagcggtcgtcgggcagcgattcgtcgcccgtcgtgggatgggccgcctcgcgcttgatcgggatgtgagtgtgttctggcacgtagaacgttgtgaagccatggtcgtcggcgagtttggccgcggccgccggggcgatgccccgatcactggtgaaaagcacgagcccgtaatccatgcacagaattagaacgtgttctacctctgtggagcaagcggcccccgctacgtcgacccgcagacgggccgctgagacgatcgctcctggtcgcgcctaggggccggtcgctcccgcgcacccgctcgaacgtgcgctagcgtggttgatcggtcgcgcgtaacgcaaacgcgggcaagcagtgacgtcgcgcccgacgaggtcttgaaggcactggaagcaaca

SEQ ID NO: 64 - DNA, 712 bp, Mycobacterium szulgai
gtgcggcggcgcccggccgggacgccgtgatcagcgagctcgtcggtattccagccgaagccgacgccgaggctgacccggctgccggacagatgatccagcgtggcaatgcttttggccagcgtgatcggatcatgctcgaccggcagcgccaccgcggtggacaaccggacccgagacgtcaccgcggccgcagcacccaaactcacccacgggtccagcgtgcgcatgtagcggtcatcgggcagcgacgcgtcactcgtagtgggatgggcagcctcccgcttgatcgggatgtgggtgtgttcaggcacgtagaacgtctgaaaaccgtggtcgtcggccagctttgcggccgccgccggggcaatgccgcgatcgctggtgaaaagtacaagcccgtaatccatgcaccgaattagaacgtgttctacctgcgatgagcaagcggcccggtcggccgacgagcaggtcggcccggcgcgaccagcagaacgtgcgctagcgtggttgatcgagtcgcgcaccggaaagcaaccggaagtaatcaggaggagccatgacctactcgaccggcagccccggatatccgcctgcgcagcagcccggggggtcgtacggcggcgccactcctggtgacgctcagagcaagcttccgctgtacctcagcatggcggtggccgccctcggcctggccgcgtatctcgccagc

SEQ ID NO: 65 - DNA, 802 bp, Mycobacterium tuberculosis
tcatagcaggcctcctcttgggtccacaacgcccgcatcgcctcgaggtattcgcgcagcatggtgcggcggcgtccgggtggcacaccatgatcgacgagctcgtcggtgttccagccgaacccgaccccgacgctgacccggccgtgcgacaaatgatccagcgtcgcaatgcttttcgccagcgtgatcggatcatgctcgaccggcagcgccaccgcggtggcaagccggatccgcgacgtcaccgccgatgctgctcccaggctcacccacgggtccaacgtgcgcatatagcggtcgtccggcagcgaagcgtcacccgtcgtcggatgggccgcctggcgcttgaccgggatgtgggtgtgttcgggcacgtaaaacgtgcgaaacccgtggctttcagcaagtctggcggccgcggccggggtgatgccgcggtcgctggtgaacagcacaagtccgtagtgcatgcaccgaattagaacgtgttccacctgcgccgggcaagcggccgtccagtcgttaatgtcgcgagcgccggtcgctccggcagcggcacccgaacgtgcgctagcgtggttgatcgaatcgcgtcgccgggagcacagcgtcgcactgcaccagtggaggagccatgacctactcgccgggtaaccccggatacccgcaagcgcagcccgcaggctcctacggaggcgtcacaccctcgttcgcccacgccgatgagggtgcgagcaagctaccgatgtacctgaacatcgcggtggcagtgctcggcctggctgcgtacttcgccagc

SEQ ID NO: 66 - DNA, 628 bp, Mycobacterium bovis
tcatagcaggcctcctcttgggtccacaacgcccgcatcgcctcgaggtattcgcgcagcatggtgcggcggcgtccgggtggcacaccatgatcgacgagctcgtcggtgttccagccgaacccgaccccgacgctgacccggccgtgcgacaaatgatccagcgtcgcaatgcttttcgccagcgtgatcggatcatgctcgaccggcagcgccaccgcggtggcaagccggatccgcgacgtcaccgccgatgctgctcccaggctcacccacgggtccaacgtgcgcatatagcggtcgtccggcagcgaagcgtcacccgccgtcggatgggccgcctggcgcttgaccgggatgtgggtgtgttcgggcacgtaaaacgtgcgaaacccgtggctttcagcaagtctggcggccgcggccggggtgatgccgcggtcgctggtgaacagcacaagtccgtagtgcatgcaccgaattagaacgtgttccacctgcgccgggcaagcggccgtccagtcgttaatgtcgcgagcgccggtcgctccggcagcggcacccgaacgtgcgctagcgtggttgatcgaatcgcgtcgccgggagcacagcgtcgcactgcaccagt

SEQ ID NO: 67 - DNA, 400 bp, Mycobacterium xenopi
gttcacccaccgcgagcaagcggcgccggtagaagctgcgatgacacgccagtcgccgcgagacccccgccgccaggtgcgctagcgtggatggtcgaatcgcgtcgcaacgcctgccctgacaagtcacggcgttaatggagcggtccacgcagcgtcgcgcggaagcggcgccctggggatacagcgtcgcaacacagtggcgccccaacggcactgatgcacaggagaagccatgacgtactcgcccggtagccccggatatccacccgcgcagtcccccggttcctacggcggctccccacagtcgttcgccaaatccgatgacggcgccagcaagctgcagctgtatctgaccgtcgcggtggtggcgctcggcctggcggcctacctggcgagt

SEQ ID NO: 68 - DNA, 707 bp, Mycobacterium paratuberculosis
tcgtagctggcttcctcgtcggtccacagcgcccgcatcgcttccaggtattcgcgcagcatggtgcggcgccggcccgccggcacgccgtggtcggcgagttcgtcggtgttccagccgaacccgacgccgaggctgacccggccgccggacagatggtcaagggtggcaatacttttcgccagcgtgatcgggtcgtgttcgaccggcagggccaccgcggtggacagccgcacccgcgaggtgacggcacaggccgcgcccagactgacccacgggtccagggtgcgcatgtagcggtcgtcgggcagcgacgcgtcgccggtggtcgggtgcgcggcctcccgcttgatcgggatatgcgtgtgttccggcacgtagaaggtcgcaaacccgtggtcgtcggcaagcttcgcggccgcagccggagagatgccacggtcgctggtgaaaagcacaagcccgtaatccatgcagtgaattagaacgtgttctacctctgcggggcaagctgtcgtgatacggaccgtctcgccgcgcggtcgtctgcgaagcccgcgggcaagccaatggcgacggcaccggccgtcgcacgtgcgctagcgtgggtgatcgaccgtgtcgctcgcgcagtgacgcgcctgcaagcaccgcgtcgcatcgcaaccgtggcgcccgctcggcactaaaaggcagtggaagcaaca

SEQ ID NO: 69 - DNA, 686 bp, Mycobacterium marinum
tcgtaggcggcttcctcctgcgtccacagtcgcccgcatcgcctcgaggtattcacgcaacatcgtgcggcgccgtccgggtggaacgccatggtcggcgagttcgtcggtgttccaaccgaaccccacgccgaggctgacccgtccgccggacagatgatccagcgtggcaatgctcttggccagggtgatcgggtcatgctcgacgggcagcgccaccgcagtcgacagccgtacccgcgaggtcaccgccgatgccgcgcccaaactcacccaggggtccagcgtgcgcatataacgatcgtcgggaagcgaggaatcgcccgtcgttggatgagcggcttctcgcttgattgggatatgggtgtgctcaggcacgtagaaggtgtgaaagccgtggtcgtcagcgagtctcgccgccgccgccggagcgatgccgcggtcgctggtgaaaagcacaagcccatagtccataacagaattagaacgtgttctacctcggccgggcaagcgccccccgcgccaatcggctcggcgggatcgacggaggtgatggcgctggtcgagcgggggcaggtcgccgcggcgcgagcaccggaacgtgcgctagcgtggttgttcgaatcgcgtcgcagggaccaagcgtcgcaatgcagcagcggcgccgcgacggcgcgcaagtaaca

SEQ ID NO: 70 - DNA, 685 bp, Mycobacterium ulcerans
tcgtaggcggcttcctcctgcgtccacagcgcccgcatcgcctcgaggtattcacgcaacatcgtgcggcgccgtccgggtggaacgccatggtcggcgagttcgtcggtgttccaaccgaaccccacgccgaggctgacccgtccgccggacagatgatccagcgtggcaatgctcttggccagggtgatcgggtcatgctcgacgggcagcgccaccgcagtcgacagccgtacccgcgaggtcaccgccgatgccgcgcccaaactcacccaggggtccagcgtgcgcatataacgatcgtcgggaagcgaggaatcgcccgtcgttggatgagcggcttctcgcttgattgggatatgggtgtgctcaggcacatagaaggtgtgaaagccgtggtcgtcagcgagtctcgccgccgccgccggagcgatgccgcggtcgctggtgaaaagcacaagcccatagtccataacagaattagaacgtgttctacctcggccgggcaagcgccccccgcgccaatcggcttggcgggatcgacggaggtgatggcgctggtcgagcgggggcaggtcgccgcggcgcgagcaccggaacgtgcgctagcgtggttgttcgaatcgcgtcgcagggaccaagcgtcgcaatgcagcagcggcgccgcgacggcgcgcaagtaaca

SEQ ID NO: 71 - DNA, 729 bp, Mycobacterium leprae
tcatataacggcttcattcttgtgtccataatgcctgcattgcttcgaggcattcgtacaccatggtgcggcgccgcccggatggcacatcgtgatcggtgagctcgttggtcttccaaccgaacccgacgccgaagttcactcactcgccggacaaattatccaggttgacaatacttttcgcaagtgtgattgggtcatgttagacgggcagcgccaccaccatgaacagtcgtagcctgccgatataacccgcatgtcgcgcccaaacttacccatgagtcataggtacgcatcgcatatagctgtcgtcactggacagtgatactcatccgtaaccaggtagtggggtctgagtggcaatggcatatgggtgtgttcgggcacatagaacttgcggaagccgtggctctccgcaagcttgactgctgccgcgggggtgatgccgcggtcgttggttaaaagcgcaatcccgtagcccataccaagaatttagagcgtgttccacctgcgacggccaagcggtcgtgccgacgatttcggcgtccatcggtggtaggcgagctgacacgcaggtcgtgccggcgcggtcgccctaacgtgcgctagcgttgatgatcgaatgcgccgcaacgtaagcgctgccaatttgggcgtttatccaacggtgcgcatgggagcacagcgttgcactgcagcagtggcgccgtgacggcactggaaataaca

SEQ ID NO: 72 - DNA, 129 bp, Mycobacterium nonchromogenicum
gttcctgttcggcgggcaacgggggggtccttgtcgcgcagtgttgacccaccgactcggcccgcaagtgcgctagcgtggatggtcgaagcgcgccgcaccgcccaccagcgccctgccacaagcaca

SEQ ID NO: 73 - DNA, 219 bp, Mycobacterium scrofulaceum
gttctacctccggtgagcaagctgccgccgcggcggcacggatcggcgtccaagccggtcgcgacggcacgcccgtcccgaagtgcgctagcgtggttgatcgatcgcgtcgcaacgcaaccgccgggcacggcattcgtggaacggcgcgcccgcacgcacagcgccgcgacgcaactgtggcgcccgcaaaggcacttcacggcactggaagcaaca

SEQ ID NO: 74 - DNA, 116 bp, Mycobacterium triplex
gttctaccttggtcggcaagcggcgcgggaacggccccggcaccggctccccgacgtgcgctagcgtggttgttcgaatcgcgtcgcaacgcaagcgcggcgagcctggaaaaaca

SEQ ID NO: 75 - DNA, 568 bp, Mycobacterium paratuberculosis
gatctcagacagtggcaggtggcggctccgaagctggcgtcagctattggtgtaccgaatgttgttgtcaccgagccggtcccaggtgtgttcgagttgcagctgagaattgtcgatccgcttagttcgccgcttgaatggtcgtctgtgccagccgcccactcgtggtctctgagtttgggtatcgatgaaatgggcgtctaccagtcgctcccgttggcgaacgtatcgggcgttgtagtgggaggcgtaccagggtcggggaaaaccgcgtggctgacgagtgctctggggtcgttcggtgcgtcagcggcggtccagttcgctgtcatcgacgggaagggtggtcaggacttggaatgcctgcgtgctcgtagctgccgattcatgaatgacgatctggagctgcctgagattgcagcgattctgaatgacgcgaccggtctagtccgtgatcgaattagacagggcaacaacatattcggatcgtccaacttttgggatcgcggcccgacgccgcaggttccgctggtgttcgtggtgattgacggctatcggggccgagatc

SEQ ID NO: 76 - DNA, 715 bp, Pseudomonas aeruginosa
gcccgtcacaccatgggagtgggttttaccagaagtggctagtctaaccgcaaggaggacggtcaccacggtaggattcatgactggggtgaagtcgtaacaaggtagccgtatcggaaggtgcggctggatcacctcctttccagagcttctcgcacaagttgagcgctcacgcttatcggctgtaaattaaagacagactcaggggtctgtagctcagtcggttagagcaccgtcttgataaggcgggggtcgttggttcgaatccaaccagacccaccattgtctgtcggtaacacacctgaggcaaatctgtacatgggggcatagctcagctgggagagcacctgctttgcaagcaggggtcgtcggttcgatcccgtctgcctccaccaatcaccaacgctaagggcttggttcagacactgaaccgagaattttgcattggcgattgagccagtcagaggatatcaacagatatcggctgtcgttctttaacaatctggaagaagtaagtaatttggatagcggaagcgtcttgagatggacgtggaaactatccgggttgtgattgtatcgatgtatctcaagatgattcgaactctaagtttgactcaattggaatacggcacaacgcgagaactcaacctgtaacgagacagactcgttatagggtcaagcgaacaagtgcatgtggtggatgccttggcrrtca

SEQ ID NO: 77 - DNA, 653 bp, Burkholderia cepacia
gcccgtcacaccatgggagtgggttgctccagaagtagctagtctaaccgcaagggggacggttaccacggagtgattcatgactggggtgaagtcgtaacaaggtagccgtaggggaacctgcggctggatcacctccttaatcgaagatctcagcttcttcataagctcccacacgaattgcttgattcactggttagacgattgggtctgtagctcagttggttagagcgcacccctgataaggtgaggtcggcagttcgaatctgcccagacccaccaattgttggtgtgctgcgtgatccgatacgggccatagctcagctgggagagcgcctgctttgcacgcaggaggtcaggagttcgatcctccttggctccaccatctaaaacaatcgtcgaaagctcagaaatgaatgttcgtgaatgaacattgatttctggtctttgcaccagaactgttctttaaaaattcgggtatgtgatagaagtaagactgaatgatctctttcactggtgatcattcaagtcaaggtaaaatttgcgagttcaagcgcgaattttcggcgaatgtcgtcttcacagtataaccagattgcttggggttatatggtcaagtgaagaagcgcatacggtggatgccttggcrrtca

SEQ ID NO: 78 - DNA, 600 bp, Pseudomonas putida
gggttccccgaagtagctagtctaaccttcgggaggacggttaccacggtgtgattcatgactggggtgaagtcgtaacaaggtagccgtaggggaacctgcggctggatcacctccttaatcgacgacatcagcctgctgatgagctcccacacgaattgcttgattcattgtcgaagacgatcaagaccctatataggtctgtagctcagttggttagagcgcacccctgataagggtgaggtcggcagttcaaatctgcccagacctaccaatatgcggggccatagctcagctgggagagcgcctgccttgcacgcaggaggtcagcggttcgatcccgcttggctccaccactcgctttacttgatcagaacttagaaatgaacattcgttgatgaatgttgatttctgacttttgtcagatcgttctttaaaaattcggatatgtgatagaaatagactgaacaccagtttcactgctggtggatcaggctaaggtaaaatttgtgagttctgctcgaaagagcaacgtgcgaattttcggcgaatgtcgtcttcacagtataaccagattgcttggggttatatggtcaagtg

SEQ ID NO: 79 - DNA, 446 bp, Pseudomonas putida
ggttcaccagaagtagctagtctaaccttcgggaggacggttaccacggtgtgattcatgactggggtgaagtcgtaacaaggtagccgtaggggaacctgcggctggatcacctccttaatcgacgacatcagcctgctgatgagctcccacacgaattgcttgattctttgtaaaagacgatcaaggccttgtgcaggcctcgcgttgttcctgatcagaacttggaaatgagcattcgcttcgaatgttgatttctggcttttgtcagatcgttctttaaaaattcggatatgtgatagaaatagactgaacaccagtttcactgctggtggatcaggctaaggtaaaatttgtgagttctgctcgaaagagcaacgtgcgaattttcggcgaatgtcgtcttcacagtataaccagattgcttggggttatatggtcaagtg

SEQ ID NO: 80 - DNA, 660 bp, Pseudomonas aeruginosa
gcccgtcacaccatgggagtgggttgctccagaagtagctagtctaaccgcaagggggacggttaccacggagtgattcatgactggggtgaagtcgtaacaaggtagccgtaggggaacctgcggctggatcacctccttaatcgaagatctcagcttcttcataagctcccacacgaattgcttgattcactggttagacgattgggtctgtagctcagttggttagagcgcacccctgataaggtgaggtcggcagttcgaatctgcccagacccaccaattgttggtgtgctgcgtgatccgatacgggccatagctcagctgggagagcgcctgctttgcacgcaggaggtcaggagttcgatcctccttggctccaccatctaaaacaatcgtcgaaagctcagaaatgaatgttcgtgaatgaacattgatttctggtctttgcaccagaactgttctttaaaaattcgggtatgtgatagaagtaagactgaatgatctctttcactggtgatcattcaagtcaaggtaaaatttgcgagttcaagcgcgaattttcggcgaatgtcgtcttcacagtataaccagattgcttggggttatatggtcaagtgaagaagcgcatacggtggatgccttggcrrtcasaggcga

SEQ ID NO: 81 - DNA, 722 bp, Burkholderia cepacia
gcccgtcacaccatgggagtgggttttaccagaagtggctagtctaaccgcaaggaggacggtcaccacggtaggattcatgactggggtgaagtcgtaacaaggtagccgtatcggaaggtgcggctggatcacctcctttccagagcttctcgcacaagttgagcgctcacgcttatcggctgtaaattaaagacagactcaggggtctgtagctcagtcggttagagcaccgtcttgataaggcgggggtcgttggttcgaatccaaccagacccaccattgtctggcggtaacacacctgaggcaaatctgtacatgggggcatagctcagctgggagagcacctgctttgcaagcaggggtcgtcggttcgatcccgtctgcctccaccaatcaccaacgctaagggcttggttcagacactgaaccgagaattttgcattggcgattgagccagtcagaggatatcaacagatatcggctgtcgttctttaacaatctggaagaagtaagtaatttggatagcggaagcgtcttgagatggacgtggaaactatccgggttgtgattgtatcgatgtatctcaagatgattcgaactctaagtttgactcaattggaatacggcacaacgcgagaactcaacctgtaacgagacagactcgttatagggtcaagcgaacaagtgcatgtggtggatgccttggcrrtcasaggcga

SEQ ID NO: 82 - DNA, 725 bp, Stenotrophomonas maltophilia
gcccgtcacaccatgggagtttgttgcaccagaagcaggtagcttaaccttcgggagggcgcttgcacggtgctgcgatgactggggtgaagtcgtaacaaggtagccgtatcggaaggtgcggctggatcacctccttttgagcaaagacagcatcgtcctgtcgggcgtcttcacaaagtacctgcattcagagaatcacaacggccaggccgatgtgagagtcccttttgggccttagctcagctgggagagcacctgctttgcaagcaggggtcgtcggttcgatcccgacagctccaccatgttcgagctgtataccgaagtccctttcgaagagcccgcacatccatgtgctactttttgaaaaagcctttcgggtctgtagctcaggtggttagacgcaccctgataagggtgaggtcggtagttcgagtctacccagacccaccattctctgaatgacgcatacattcgatctttatacgcatcagcactgtggctggtacgtgttcttttaaaacttgtgacgtagcgagcgtttgagatgttctatcagacgtgtcgtgaggctaaggcgagagacgcaagtctctttattgattgagtcgttatattcgtatccgggctttgtacccccgggtcgtgtgtaacccaaggcaacttgcggttatatggtcaagcgaataagcgcacacggtggatgccttggcrrtcasaggcga

SEQ ID NO: 83 - DNA, 18 bp, Pseudomonas sp.
acgtcacaccatgggagt

SEQ ID NO: 84 - DNA, 23 bp, Burkholderia cepacia
ccctgagtctgtctttaatttac

SEQ ID NO: 85 - DNA, 20 bp, Pseudomonas aeruginosa
ctttcgacgattgttttagt

SEQ ID NO: 86 - DNA, 21 bp, Stenotrophomonas maltophilia
tcaataaaagagacttgcgtc

SEQ ID NO: 87 - DNA, 18 bp, Pseudomonas sp.
gattgccaaggcatccac

SEQ ID NO: 88 - DNA, 18 bp, Pseudomonas sp.
gaggaaggtggggatgac

SEQ ID NO: 89 - DNA, 18 bp, Pseudomonas sp.
tgggaacgtattcaccgt